(19) United States
(12) Patent Application Publication
(10) Pub. No.: US 2008/0194673 A1
(43) Pub. Date: Aug. 14, 2008

(54) Environment-Friendly Pollution-Proof Agent

(76) Inventor: Hyun Woung Shin, Chungcheongnam-do (KR)

Correspondence Address: Ronald R. Santucci, Frommer Lawrence & Haug, LLP., 745 Fifth Avenue, NY, 10151, US

(21) Appl. No.: 11/629,814

(22) PCT Filed: May 23, 2005
(86) PCT No.: PCT/KR2005/001503

§ 371 Date: Sep. 26, 2007

(30) Foreign Application Priority Data
Jun. 18, 2004 (KR) 10-2004-0045531
Jun. 23, 2004 (KR) 10-2004-0047048
Sep. 22, 2004 (KR) 10-2004-0075837

(51) Int. Cl. C09D 5/16; C07D 309/00; A01N 27/00; A01N 37/00; A01N 43/16

(52) U.S. Cl. 514/451; 106/15.05; 549/356; 523/122; 514/762; 514/529

(57) ABSTRACT

The present invention relates to an environmental friendly antifouling agent, and more particularly, to a novel Sargassum-derived antifouling agent which is harmless to environment, has antifouling activity against a broad spectrum of fouling organisms, can be extracted from nature, resulting in a relative reduction in production cost as compared with the existing antifouling substances, and can effectively prevent the pollution of marine environment caused by the use of toxic antifouling agents, such as TBT.

### TECHNICAL FIELD

The present invention relates to an environmental friendly antifouling agent extracted from Sargassum, and more particularly, to a novel antifouling agent extracted from brown alga, Sargassum, which is harmless to environment, has antifouling activity against a broad spectrum of fouling organisms, can be extracted from nature, resulting in a relative reduction in production cost as compared with the existing antifouling substances, and can effectively prevent the pollution of marine environment caused by the use of toxic antifouling agents, such as TBT.

### BACKGROUND ART

An antifouling substance refers to a substance which is mixed with paints in order to prevent the fouling of the vessel surface by marine fouling organisms (microorganisms, animals and plants). Fouling means that benthic organisms attach and grow on artificial or natural objects. The attachment of benthic organisms on the vessel surface causes an increase with friction force, resulting in a reduction at the vessel speed, an acceleration of corrosion, and an increase in fuel use, leading to economic loss.

It is known that if the bottom of vessels is exposed to seawater for 6 months, fouling organisms will attach on the vessel bottom in an amount of 150 kg/m². It has also been reported that, for large-sized vessels, each when the vessel surface roughens at 0.01 mm due to the attachment of fouling organisms, the friction force will be increased by 0.3-1.0%, resulting in a reduction of about 50% at the vessel speed.

In an attempt to solve this problem, organic tin compounds, such as tributyltin (hereinafter, referred to as “TBT”), have been frequently used as antifouling agents. However, as the fact that TBT adversely affects marine environment is found, the Marine Environment Protection Committee (MEPC) of the International Maritime Organization (IMO), which is the UN specialized agency, adopted a resolution of the restriction of antifouling systems for vessels due to the risk of organic tin compounds. As a result, the use of TBT as an antifouling agent was entirely prohibited from Jan. 1, 2003, and a regulation that TBT should be removed from vessels will become effective from the year 2008.

Tin-free antifouling substances, which are currently used as substitutes for organic tin compounds, include cuprous oxide and zinc, as disclosed in Korean patent laid-open publication No. 2001-0099049. The tin-free antifouling agents have a technical problem in that they are insufficient in an antifouling effect against algae, such as green algae. Also, the cuprous oxide antifouling agent also adversely affects environments due to accumulation on the marine bottom, and thus its use will be prohibited between 2006 and 2008. Accordingly, there is an urgent need for the development of an antifouling agent which is excellent in antifouling effect and at the same time, harmless to environment.

Thus, the present inventors have conducted many studies to solve the above-described problems and to develop an antifouling agent which is harmless to environment, excellent in antifouling effect and low in production cost, and consequently, found that substances extracted from Sargassum have excellent antifouling activity, thereby completing the present invention.

### DISCLOSURE OF INVENTION

It is therefore an object of the present invention to provide a novel antifouling agent which is not only low in production cost since it is extracted from natural materials but also environmental friendly as it is extracted from Sargassum.

Another object of the present invention is to provide an environmental friendly antifouling paint extracted from Sargassum.

Still another object of the present invention is to provide an environmental friendly biocide extracted from Sargassum.

To achieve the above objects, in one aspect, the present invention provides an antifouling agent containing an extract from Sargassum as an active ingredient.

Also, in another aspect, the present invention provides an antifouling agent containing as active ingredients one or two or more compounds selected from the group consisting of hexadecane, octadecane, eicosane, 1-eicosanol, 1-pentadecanol, dibutyl phthalate, dioctyl phthalate, diisononyl phthalate, dicyclohexyl phthalate, methyl palmitate, methyl stearate, methyl linoleate, and 6-formyl-3,4-dehydro-2,8-dimethyl-2-(3′,6′-dienyl-8′-hydroxy-4′-methylnonane)-2H-1-benzopyrane.

Also, in another aspect, the present invention provides a novel 6-formyl-3,4-dehydro-2,8-dimethyl-2-(3′,6′-dienyl-8′-hydroxy-4′-methylnonane)-2H-1-benzopyrane compound which has antifouling property and is extracted from Sargassum, and a preparation method thereof.

In another aspect, the present invention provides an antifouling paint comprising a resin, a solvent, a pigment, an antifouling substance and other additives, in which the antifouling substance is one or a mixture of two or more compounds selected from hexadecane, octadecane, eicosane, 1-eicosanol, 1-pentadecanol, dibutyl phthalate, dioctyl phthalate, diisononyl phthalate, dicyclohexyl phthalate, methyl palmitate, methyl stearate, methyl linoleate, and 6-formyl-3,4-dehydro-2,8-dimethyl-2-(3′,6′-dienyl-8′-hydroxy-4′-methylnonane)-2H-1-benzopyrane.

Also, the above-described Sargassum extract can be used as not only antifouling agents but also biocides since it has an algae inhibitory effect. Thus, these antifouling agents and biocides are within the scope of the present invention.

Hereinafter, the present invention will be described in more detail.

The present invention relates to an environmental friendly antifouling agent extracted from Sargassum, and more particularly, to a novel antifouling agent extracted from Sargassum, which is harmless to environment, has antifouling activity against a broad spectrum of fouling organisms, can be extracted from nature, resulting in a relative reduction in production cost as compared with the existing antifouling substances, and can effectively prevent the pollution of marine environment caused by the use of toxic antifouling agents, such as TBT.

In order to develop an environmentally harmless antifouling paint, the present inventors have tested the antifouling activity of various kinds of seaweeds and land plants. For use as the seaweeds, species inhabiting the intertidal of various areas of the Korean east and south coasts and species collected by diving were screened, identified, dried in the shade, and crushed with a crusher, and the powder sample was stored in a glass bottle and used when required. For use as the land plants, among land plants inhabiting the whole regions of Korea, plants whose secondary metabolic products are known to be able to have antifouling activity by literature information were collected, screened, identified, dried in the shade, crushed with a crusher, and extracted.

The effect of the extracts on the inhibition of the attachment of fouling algae was tested using the spores of Enteromorpha prolifera and Ulva pertusa, which are typical fouling algae. In the test, an extract from Sargassum among various algae and land plants showed excellent antifouling activity.

The present inventors found from a Sargassum extract having excellent antifouling property through various experiments that the substances exhibiting antifouling activity are hexadecane, octadecane, eicosane, 1-eicosanol, 1-pentadecanol, dibutyl phthalate, dioctyl phthalate, diisononyl phthalate, dicyclohexyl phthalate, methyl palmitate, methyl stearate, methyl linoleate, and 6-formyl-3,4-dehydro-2,8-dimethyl-2-(3′,6′-dienyl-8′-hydroxy-4′-methylnonane)-2H-1-benzopyrane.

Specifically, in the present invention, a novel 16-formyl-3,4-dehydro-2,8-demethyl-2-(3′,6′-dienyl-8′-hydroxy-4′-methylnonane)-2H-1-benzopyrane compound was found from Sargassum confusum. This compound was prepared by crushing Sargassum confusum to make Sargassum confusum powder, extracting the Sargassum confusum powder with one solvent selected from the group consisting of hexane, ethyl acetate and methanol and collecting the supernatant, and vacuum-concentrating the collected supernatant.

Meanwhile, an antifouling paint generally comprises an antifouling substance, a resin, a solvent, a pigment, other additives and the like, and may also contain a booster for improving antifouling activity.

The paint according to the present invention contains the antifouling substance in an amount of 3-40% by weight, and preferably 10-30% by weight. If the content of the antifouling substance is less than 3% by weight, the paint will be insufficient in antifouling activity, and if the content of the antifouling substance is more than 30% by weight, the mixing properties with other components, and long-term storage properties will be deteriorated.

Resins which can be used in the inventive antifouling paint include all the resins used in the prior antifouling paints. Examples thereof include vinyl resins, such as vinyl acetate and vinyl chloride resins, synthetic resins, such as urethane, rubber chloride, phthalic acid, alkid, epoxy, phenol, melanine, acrylic, fluorine and silicon resins, and natural resins, such as rosin. Particularly, the acrylic resin is a polymer containing one or two or more monomers selected from, for example, w-(N-isothiazolonyl) alkyl acrylate, w-(N-isothiazolonyl) alkyl methacrylate, w-(N-4-chloroisothiazolonyl) alkyl acrylate, w-(N-4-chloroisothiazolonyl) alkyl methacrylate, w-(N-5-chloroisothiazolonyl) alkyl acrylate, w-(N-5-chloroisothiazolonyl) alkyl methacrylate, w-(N-4,5-dichloroisothiazolonyl) alkyl acrylate, w-(N-4,5-dichloroisothiazolonyl) alkyl methacrylate, w-maleimidoalkyl acrylate, w-maleimidoalkyl methacrylate, w-2,3-maleimidoalkyl acrylate, w-2,3-dichloromaleimidoalkyl methacrylate, w-maleimidoalkyl vinyl ether, 4-maleimidoalykl acrylate, acrylic acid, methacrylic acid, maleic anhydride, hydroxylalkyl acrylate, alkoxyalkyl acrylate, phenoxyalkyl acrylate, w-(acetoacetoxy) alkyl acrylate, w-(acetoacetoxy) alkyl metacrylate, w-(acetoacetoxy) alkyl vinyl ether, vinyl acetoacetoacetate, N,N′-dialkylacrylate, vinylpyridine, vinylpyrrolidone, 4-nitrophenyl-2-vinyl ethylate, 2,4-dinitrophenyl acrylate, 2,4-dinitrophenyl methacrylate, 4-thiocyanophenyl acrylate, trialkylsilyl acrylate, monoalkyldiphenylsilyl acrylate, dichloromethyl acrylate, chloromethyl methacrylate, phenylmethyl acrylate, diphenylmethyl acrylate, diphenylmethyl methacrylate, zinc trialkyl acrylate, zinc triakyl methacrylate, zinc triaryl acrylate, zinc triaryl methacrylate, copper trialkyl acrylate, copper trialkyl methacrylate, copper triaryl acrylate, and copper triaryl methacrylate. A preparation method of this polymer is well described in Korean patent application No. 95-15149. The number-average molecular weight of the polymer is preferably in a range of 1,500-100,000 in view of viscosity, film formation ability and workability. Moreover, the synthetic resin may also be used in combination with the natural resin. The content of the resin in the paint is 2-20% by weight, and preferably 5-15% by weight. If the resin content is less than 2% by weight, the adhesion of the paint will be reduced, and if the content is more than 20% by weight, the paint will have a problem in storage properties.

Solvents which can be used in the inventive antifouling paint include hydrocarbon and ketone solvents, such as xylene, methylethylketone and methylisobutylketone, and cellosolve acetate, and preferably used in an amount of 10-30% by weight. If the solvent content is less than 10% by weight, the paint will have excessively high viscosity, and if the solvent content is more than 30% by weight, the paint will have problems in adhesion and antifouling activity.

Pigments which can be used in the inventive antifouling paint include various pigments known in the art. For example, metal oxides, such as titanium oxide, iron oxide and zinc oxide, and organic pigments, may be used alone or in a mixture. The pigment is preferably used in an amount of 20-40% by weight. If the pigment is used in an amount of less than 20% by weight, the problem of discoloration will occur, and if it is used in an amount of more than 40% by weight, the weather resistance of the paint will be deteriorated.

If the antifouling activity of the paint needs to be further improved, a booster can be used. Examples thereof include zinc pyrithione, copper pyrithione, polyhexamethyleneguanidine phosphate, 2,4,5,6-terachloro-isophthalonitrile, 3-(3,4-dichlorophenyl)-1,1-dimethylurea, 2-methylthio-4-terbutylamino-6-cyclopropylamino-s-triazine, zinc ethylenebisdithiocarbamate, manganese ethylenebisdithiocarbamate, 2-n-octyl-4,5-dichloro-2-methyl-4-isothiazoline-3-one, 2-(thiocyanomethylthio) benzothiazole, 2,3,5,6-tetrachloro-4-(methylsulphonyl)pyridine, 3-iodo-2-propynyl butylcarbamate, diiodomethyl-p-tolylsulfone, 1,2-benzoisothiazolin-3-one, 2-methylthio-4-tert-butylamino-6-cyclopropylamino-s-triazine, 2-(4-thiocyanomethylthio) benzothiazole), 2-n-octyl-4-isothiazolin-3-one, N-(fluorodichloromethylthio)-phthalimide, N-dichlorofluoromethylthio-N′,N′-dimethyl-N-p-tolylsulfamide, N,N-dimethyl-N′-phenyl-(fluorodichloromethylthio)-sulphamide, zinc(2-pyridylthio-1-oxide), copper (2-pyridylthio-1-oxide), and silver compounds. These compounds may be used alone or a mixture of two or more. The booster is preferably used in an amount of 1-7% by weight, and more preferably 2-4% by weight. If the booster content is used in an amount of less than 1%, its effect on an increase in antifouling activity will be insignificant, and if it is used in an amount of more than 7%, the paint will have a problem in storage stability.

In addition, the inventive paint composition may also contain various known additives. Examples of the additives may include thickeners, such as polyamide wax, bentonite or polyethylene wax. These additives are preferably used in an amount of 1-5% by weight. If the content of the additives is more than 5% by weight, the viscosity of the paint will be excessively increased.

Furthermore, the Sargassum extract according to the present invention, and one or two or more compounds selected from the following compounds may also be used in biocides since they have algae movement inhibitory activity: hexadecane, octadecane, eicosane, 1-eicosanol, 1-pentadecanol, dibutyl phthalate, dioctyl phthalate, diisononyl phthalate, dicyclohexyl phthalate, methyl palmitate, methyl stearate, methyl linoleate, and 6-formyl-3,4-dehydro-2,8-demethyl-2-(3′,6′-dienyl-8′-hydroxy-4′-methylnonane)-2H-1-benzopyrane. The biocide may preferably contain as active ingredients the inventive extracts or compounds in an amount of 0.1-5% by weight based on the total weight of the biocide. In addition to the active ingredients, the biocide may also contain a surfactant, a solvent, and an isothiazolone biocide.

### BRIEF DESCRIPTION OF DRAWINGS

FIG. 1 shows the TLC diagram at 365 nm of a hexane extract of Sargassum confusum.

FIG. 2 shows the TLC diagram showing 2454 nm of a hexane extract of Sargassum confusum

FIG. 3 shows the HPLC profile for V-e-K7 fraction.

FIG. 4 shows the profile of GC-Mass spectrum of 6-formyl-3,4-dehydro-2,8-dimethyl-2-(3′,6′-dienyl-8′-hydroxy-4′-methylnonane)-2H-1-benzopyrane.

FIG. 5 shows the TLC diagram for fractions V-i˜V-vi.

FIG. 6 shows the TLC diagram for fractions V-iii-a˜V-iii-c.

FIG. 7 shows the HPLC chromatogram of the fraction V-iii-a.

FIG. 8 shows ¹H NMR data for the fraction A of the second embodiment.

FIG. 9 shows ¹H NMR data for the fraction B of the second embodiment.

FIG. 10 shows ¹H NMR data for the fraction C of the second embodiment.

FIG. 11 shows ¹H NMR data for the fraction D of the second embodiment.

FIG. 12 shows ¹H NMR data for the fraction E of the second embodiment.

FIG. 13 shows ¹H NMR data for the fraction F of the second embodiment.

FIG. 14 shows ¹H NMR data for the fraction G of the second embodiment.

FIG. 15 shows the profile of ¹H NMR spectrum of 6-formyl-3,4-dehydro-2,8-demethyl-2-(3′,6′-dienyl-8′-hydroxy-4′-methylnonane)-2H-1-benzopyrane.

FIG. 16 shows the profile of ¹³C NMR spectrum of ¹H NMR spectrum of 6-formyl-3,4-dehydro-2,8-demethyl-2-(3′,6′-dienyl-8′-hydroxy-4′-methylnonane)-2H-1-benzopyrane.

FIG. 17 shows the two-dimensional NMR spectra of 6-formyl-3,4-dehydro-2,8-dimethyl-2-(3′,6′-dienyl-8′-hydroxy-4′-methylnonane)-2H-1-benzopyrane.

FIG. 18 shows the HMBC correlation of 6-formyl-3,4-dehydro-2,8-dimethyl-2-(3′,6′-dienyl-8′-hydroxy-4′-methylnonane)-2H-1-benzopyrane.

FIG. 19 shows an example of a partial pattern by a mass of 6-formyl-3,4-dehydro-2,8-dimethyl-2-(3′,6′-dienyl-8′-hydroxy-4′-methylnonane)-2H-1-benzopyrane.

FIG. 20 shows the profile of IR spectrum of 6-formyl-3,4-dehydro-2,8-dimethyl-2-(3′,6′-dienyl-8′-hydroxy-4′-methylnonane)-2H-1-benzopyrane.

FIGS. 21 to 23 show GC-MS data of NMR data for K4 fraction of the fourth embodiment.

FIGS. 24 to 26 show NMR data of NMR data for K4 fraction of the fourth embodiment.

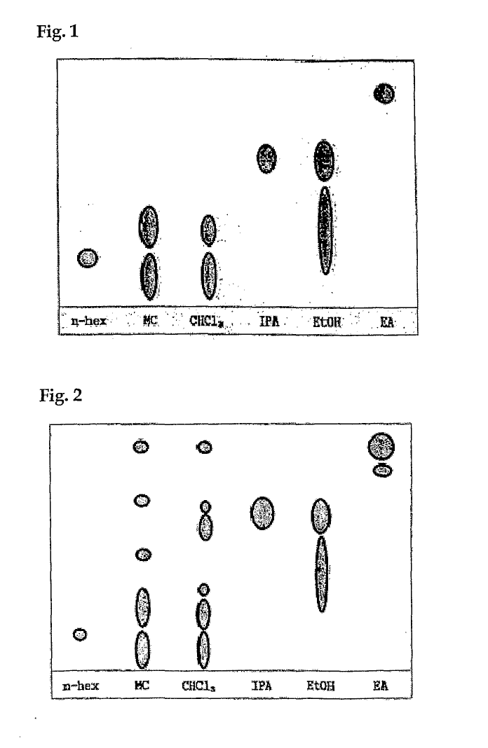

### BEST MODE FOR CARRYING OUT THE INVENTION

The present invention will hereinafter be described in further detail by examples. It will however be obvious to a person skilled in the art that the present invention is not limited to or by these examples.

### EXAMPLE 1

### Sample Collection and Extraction

1) Extraction of Sargassum confusum

Sargassum confusum was collected in Pohang (located at a latitude of 36 degrees, a longitude of 129 degree 30 minutes), Gyeongsangbuk-do, Korea, and foreign materials were removed from the marine algae. Then, the marine algae were washed, dried in the shade. In order to increase extraction yield, the land plants were powdered with a crusher and used in tests. 1 kg of the sample powder was added to 10 liters of hexane, stored for 24 hours and extracted, and only the supernatant was collected. The extraction and supernatant collection procedures were repeated three times, and the supernatants were combined together, and then, evaporated to a volume of 1/10 with a vacuum concentrator at 37° C. The remaining material was filtered through a 0.22-μm filter and used in tests with storage at −20° C.

Identification of Solubility for Organic Solvents

In order to check solubility of Sargassum confusum hexane extracts for organic solvents, the solubility of hexane, chloroform, methylene chloride, ethyl acetate, ethanol, methanol was identified by a UV lamp (UVGL-58, UV-254/366, UVP Inc. U.S.A) using the thin layer chromatography (TLC) (SIL G/UV254, 0.25 mm layer with fluorescent indicator, Machereynagel Co. Germany).

2) Extraction of Sargassum sp.

The inhibitory effect of organism-derived extracts against the attachment of spore of Enteromorpha prolifera was tested. Each of methyl alcohol extracts of samples was tested for attachment inhibitory effect at a concentration of 200 μl/ml, and as a result, extracts of Sargassum sp. (139), Sargassum sp. (365) and Sargassum sp. (383) showed the most excellent inhibitory effect against the attachment of spores (see Table 1a, 1b).

TABLE 1a

| | Spore attachment (%) | |
|---|---|---|
| | Methanol extracts | Water extracts |
| Species | (200 mg/ml) | (200 mg/ml) |
| Lomentaria catenata | 86 ± 7 | 79 ± 4 |
| Sargassum confusum | 75 ± 4 | 78 ± 5 |
| Identifying | 85 ± 7 | 85 ± 9 |
| Laminaria sp. | 77 ± 8 | 84 ± 5 |
| Sargassum thunbergii | 55 ± 9 | 76 ± 8 |
| Sargassum fulvellum | 45 ± 11 | 87 ± 4 |
| Ishige okamurai | 85 ± 8 | 85 ± 4 |
| Sargassum sp. | 86 ± 9 | 88 ± 6 |
| Sargassum sp. | 69 ± 9 | 84 ± 6 |
| Sargassum sp. (139) | 21 ± 5* | 84 ± 5 |
| Sargassum sp. | 72 ± 7 | 71 ± 9 |
| Sargassum sp. ringggoidianum | 79 ± 6 | 79 ± 3 |
| Identifying | 78 ± 8 | 107 ± 6 |
| Ecklonia kurome | 54 ± 5 | 95 ± 7 |
| Identifying | 86 ± 6 | 81 ± 7 |
| Identifying | 95 ± 2 | 78 ± 8 |
| Sargassum filicinum | 72 ± 7 | 71 ± 8 |
| Sargassum filicinum | 91 ± 8 | 86 ± 9 |
| Identifying | 87 ± 4 | 95 ± 8 |
| Ishige okamurai | 92 ± 6 | 76 ± 6 |
| Gelidium amansii | 88 ± 4 | 95 ± 5 |
| Chondria crassiculis | 88 ± 6 | 71 ± 6 |
| Sargassum thunbergii | 88 ± 3 | 85 ± 2 |
| Chondrus ocellatus | 81 ± 4 | 85 ± 2 |
| Sargassum sp. | 48 ± 6 | 96 ± 8 |
| Sargassum sp. | 76 ± 8 | 74 ± 9 |
| Sargassum horneri | 85 ± 2 | 94 ± 6 |
| Pachymeniopsis elliptica | 94 ± 1 | 78 ± 8 |
| Gelidium amansii | 70 ± 7 | 86 ± 8 |
| Identifying | 69 ± 3 | 84 ± 6 |
| Gracilaria verrucosa | 81 ± 8 | 87 ± 7 |
| Identifying | 86 ± 4 | 73 ± 8 |
| Identifying | 77 ± 6 | 79 ± 10 |
| Grateloupia filicina | 95 ± 5 | 87 ± 4 |

TABLE 1b
(continued)

| | Spore attachment (%) | |
|---|---|---|
| | Methanol extracts | Water extracts |
| Species | (200 mg/ml) | (200 mg/ml) |
| Gelidium amansii | 91 ± 7 | 94 ± 8 |
| Identifying | 74 ± 9 | 86 ± 5 |
| Pachymeniopsis sp. | 83 ± 8 | 93 ± 6 |
| Chondrus ocellatus | 89 ± 2 | 89 ± 9 |
| Ecklonia cava | 87 ± 4 | 79 ± 7 |
| Grateloupia sp. | 85 ± 7 | 77 ± 4 |
| Lomentaria catenata | 87 ± 11 | 85 ± 7 |
| Corallina sp. | 77 ± 6 | 95 ± 5 |
| Gymnogongrus flabelliformis | 74 ± 5 | 96 ± 8 |
| Gelidium amansii | 74 ± 5 | 86 ± 8 |
| Identifying | 68 ± 6 | 89 ± 6 |
| Identifying | 89 ± 5 | 87 ± 7 |
| Sargassum horneri | 95 ± 4 | 92 ± 2 |
| Ulva pertusa | 92 ± 8 | 85 ± 2 |
| Identifying | 81 ± 9 | 91 ± 3 |
| Identifying | 79 ± 1 | 64 ± 8 |
| Sargassum sp. | 64 ± 3 | 69 ± 9 |
| Sargassum sp. | 78 ± 5 | 86 ± 4 |
| Agarum cribrosum | 91 ± 7 | 85 ± 7 |
| Sargassum horneri | 93 ± 8 | 94 ± 8 |
| Identifying | 91 ± 8 | 92 ± 9 |
| Identifying | 89 ± 8 | 76 ± 9 |
| Chondrus ocellatus | 85 ± 9 | 75 ± 5 |
| Carpopeltis cornea | 77 ± 6 | 86 ± 4 |
| Chondrus sp. | 60 ± 4 | 74 ± 7 |
| Codium sp. | 91 ± 4 | 72 ± 8 |
| Pachymeniopsi lanceolata | 92 ± 4 | 84 ± 9 |
| Chondrus sp. | 95 ± 2 | 78 ± 6 |
| Identifying | 84 ± 7 | 77 ± 6 |
| Sargassum sp. | 96 ± 4 | 92 ± 3 |
| Pachymeniopsi lanceolata | 79 ± 4 | 84 ± 2 |
| Ulva sp. | 85 ± 8 | 74 ± 5 |
| Carpopeltis cornea | 88 ± 9 | 69 ± 8 |
| Sargassum sp. (365) | 31 ± 9* | 82 ± 5 |
| Grateloupia okamurai | 24 ± 9* | 94 ± 4 |
| Sargassum sp. (383) | 31 ± 4* | 86 ± 3 |
| Brassica sp. | 30 ± 6* | 78 ± 9 |
| Chaetomorpha aerea | 78 ± 8 | 64 ± 6 |
| Sargassum sp. | 51 ± 7 | 54 ± 8 |

### EXAMPLE 2

### Solvent Fractionation of Sargassum confusum Extract

A glass tube column (10 cm×90 cm, equipped with PTEE end plate) was packed with silica gel (70-230 meshes) in hexane. In this regard, the column was selected and used depending on the amount of the sample, and the amount of the silica gel was 50-60 times the amount of the sample. The Sargassum confusum extract as a sample was applied to the column, and the column was developed sequentially with the following developing solvents at a flow rate of 5 ml/min: hexane, hexane:ethyl acetate=95:5, hexane:ethyl acetate=90:10, hexane:ethyl acetate=85:15, hexane:ethyl acetate:=80:20, hexane:ethyl acetate=70:30, hexane:ethyl acetate=60:40, hexane:ethyl acetate:=50:50, ethyl acetate, ethyl acetate:methanol=95:5, ethyl acetate:methanol=90:10, and ethyl acetate:methanol=80:20. The eluates were fractionated into six fractions (I-VI) by performing thin layer chromatography under the developing solvent condition.

TABLE 2

| Developing solvent | Volume (ml) |
|---|---|
| Hexane | 2000 |
| Hexane:ethyl acetate (95:5) | 2000 |
| Hexane:ethyl acetate (90:10) | 2000 |
| Hexane:ethyl acetate (85:15) | 2000 |
| Hexane:ethyl acetate (80:20) | 2000 |
| Hexane:ethyl acetate (70:30) | 2000 |
| Hexane:ethyl acetate (60:40) | 2000 |
| Hexane:ethyl acetate (50:50) | 2000 |
| Ethyl acetate | 2000 |
| Ethyl acetate:methyl alcohol (95:5) | 2000 |
| Ethyl acetate:methyl alcohol (90:10) | 2000 |
| Ethyl acetate:methyl alcohol (80:20) | 2000 |

Inhibitory Effect Test Against the Mobility of the Spores of Ulva pertusa

The effects of the fractions I-VI on the inhibition of attachment of fouling organisms were tested on Ulva pertusa, one of typical fouling algae. Ulva pertusa was collected in Kyongpodae, Kangnung-city, Kangwon-Do, Korea. The collected seaweed was transported to a laboratory in which only Ulva pertusa was screened. In order to remove fouling organisms, the screened algae were treated three times with supersonic waves for 1 minute for each time, and then washed clean with sterilized seawater. The washed algae were simply sterilized by immersion in a mixed solution of 1% betadine and 2% trition X-100 for 1 minute, followed by semi-drying. The semi-dried Ulva pertusa was added to sterilized seawater and placed in a 80 μmol m⁻²s⁻, 20° C. incubator to induce the release of spores.

10 μl of dimethyl sulfoxide (DMSO) was placed in a prepared tube, to which the seawater having spores released therein was then added. The Inhibitory effects of the fractions against the mobility of spores at varying concentrations of 500, 1000, 1500, 2000, 4000 μg/ml were observed with a microscope (Olympus CK-2) at 100× magnification, and the results are shown in Table 3 below. In Table 3, “++++” designates more than 95% inhibition of the mobility of spores, “+++” designates 95%-75% inhibition of the mobility of spores, “++” designates 75%-50% inhibition, “+” designates 50%-20% inhibition of the mobility of spores, and “−” designates no inhibition of the mobility of spores. Before inoculation with each fraction at each dilution concentration, the movement of spores in the seawater was examined and the result was used as a comparison with a control group.

The five fractions (I-VI) were tested for inhibitory activity against the mobility of spores, and the results are shown in Table 3 below. As can be seen in Table 3, the fraction III showed strong activity. The fraction III, which show good activity in the test, was subjected to the second silica gel column chromatography. In this regard, the fraction III was applied to a column, and the column was developed sequentially with the following developing at a flow rate of 3 ml/min: hexane, hexane:ethyl acetate=95:5, hexane:ethyl acetate=90:10, hexane:ethyl acetate=85:15, hexane:ethyl acetate=80:20, hexane:ethyl acetate=70:30, hexane:ethyl acetate=60:40, hexane:ethyl acetate=50:50, ethyl acetate, ethyl acetate:methanol=95:5, ethyl acetate:methanol=90:10, and ethyl acetate:methanol=80:20. The elutes were fractionated into eight fractions (A-H) by performing thin layer chromatography in the developing solvent condition. The respective eight fractions (A-H) of Sargasuum confusum were tested for inhibitory activity against the mobility of spores, and the results are shown in Table 4 below. As can be seen in Table 4, all the eight fractions B-D showed strong activity.

TABLE 3

| Concentration | Mobility inhibitory activity | | | | | |
|---|---|---|---|---|---|---|
| (μg/ml) | I | II | III | IV | V | VI |
| 4,000 | ++++ | ++++ | ++++ | ++++ | ++++ | ++++ |
| 2,000 | ++++ | ++++ | ++++ | ++++ | ++++ | ++++ |
| 1,000 | +++ | ++++ | ++++ | ++++ | ++++ | ++++ |
| 500 | +++ | +++ | ++++ | ++++ | ++++ | +++ |
| 250 | +++ | +++ | ++++ | ++ | +++ | ++ |
| 125 | + | ++ | ++++ | ++ | +++ | ++ |

TABLE 4

| Concentration | Mobility inhibitory activity | | | | | | | |
|---|---|---|---|---|---|---|---|---|
| (μg/ml) | A | B | C | D | E | F | G | H |
| 4,000 | ++++ | ++++ | ++++ | ++++ | ++++ | ++++ | ++++ | ++++ |
| 2,000 | ++++ | ++++ | ++++ | ++++ | ++++ | ++++ | ++++ | ++++ |
| 1,000 | ++++ | ++++ | ++++ | ++++ | ++++ | ++++ | ++++ | ++++ |
| 500 | ++++ | ++++ | ++++ | ++++ | ++++ | ++++ | ++++ | ++++ |
| 250 | ++++ | ++++ | ++++ | ++++ | ++++ | ++++ | ++++ | ++++ |
| 125 | ++++ | ++++ | ++++ | ++++ | ++++ | ++++ | ++++ | ++++ |

### EXAMPLE 3

### Solvent Fractionation of Sargassum confusum Extract

A glass tube column (10 cm×90 cm, equipped with PTEE end plate) was packed with silica gel (70-230 meshes) in hexane. In this regard, the column was selected and used depending on the amount of the sample, and the amount of the silica gel was 50-60 times the amount of the sample. The Sargassum confusum extract as a sample was applied to the column, and the column was developed sequentially with the following developing solvents at a flow rate of 10 ml/min: hexane, hexane:methylene chloride=80:20, hexane:methylene chloride=60:40, hexane:methylene chloride=40:60, hexane:methylene chloride=60:40, hexane:methylene chloride=20:80, methylene chloride, methylene chloride:ethanol=80:20, methylene chloride:ethanol=60:40, methylene chloride:ethanol=40:60, methylene chloride:ethanol=20:80, ethyl acetate, ethyl acetate:methanol=90:10, ethyl acetate:methanol=80:20, ethyl acetate:methanol=70:30, ethyl acetate:methanol=60:40, ethyl acetate:methanol=50:50. The elutes were fractionated into eight fractions (I-VIII) by performing thin layer chromatography under the same developing solvent condition as in Example 2.

The effects of the land plant and algae extracts on the inhibition of attachment of fouling organisms were tested on Ulva pertusa, one of typical soft fouling algae. Ulva pertusa was collected in Kyongpodae, Kangnung-city, Kangwon-Do, Korea. The collected seaweed was transported to a laboratory in which only Ulva pertusa was screened. In order to remove fouling organisms, the screened layer was treated three times with supersonic waves for 1 minute for each time, and then washed clean with sterilized seawater. The washed algae were simply sterilized by immersion in a mixed solution of 1% betadine and 2% trition X-100 for 1 minute, followed by semi-drying.

The semi-dried Ulva pertusa was added to sterilized seawater and placed in a 80 μmol m⁻²s⁻¹, 20° C. incubator to induce the release of spores.

10 μl of dimethyl sulfoxide (DMSO) was placed in a prepared tube, to which the seawater having spores released therein was then added. The inhibitory effects of the fractions against the mobility of spores at varying concentrations of 125, 250, 500, 1000, 1500, 2000, 4000 μg/ml were observed with a microscope (Olympus CK-2) at 100× magnification, and the results are shown in Table 5 below. In Table 5, “++++” designates more than 95% inhibition of the mobility of spores, i.e., a possibility that the mobility of spores will be hardly observed, “+++” designates 95%-75% inhibition of the mobility of spores, “++” designates 75%-50% inhibition, “+” designates 50%-20% inhibition of the mobility of spores, and “−” designates less than 20% inhibition of the mobility of spores.

TABLE 5

| Concentration | Activity | | | | | | | |
|---|---|---|---|---|---|---|---|---|
| ((g/ml) | I | II | III | IV | V | VI | VII | VIII |
| 4,000 | +++ | ++ | +++ | ++++ | ++++ | +++ | ++ | +++ |
| 2,000 | ++ | ++ | +++ | ++++ | ++++ | ++ | ++ | ++ |
| 1,000 | + | ++ | +++ | ++++ | ++++ | ++ | + | + |
| 500 | + | + | ++ | ++++ | ++++ | + | + | + |
| 250 | − | − | ++ | +++ | +++ | − | − | + |
| 125 | − | − | ++ | ++ | +++ | − | − | − |

As shown in Table 5, in the results of an inhibitory activity test against the mobility of Enteromorpha spores, the fraction V showed the most powerful inhibitory activity at a concentration of 4,000 (g/ml.

The fraction V was developed on Prep-TLC in a mixed solvent of hexane:methylene chloride:ethyl acetate (3:1:1) to collect five fractions, i.e., V-a, V-b, V-c, V-d and V-e.

Each of the five fractions was tested for an inhibitory effect against the mobility of Enteromorpha spores, and the results are shown in Table 6 below.

TABLE 6

| Concentration | | Activity | | | | |
|---|---|---|---|---|---|---|
| | ((g/ml) | V-a | V-b | V-c | V-d | V-e |
| | 4,000 | ++++ | ++++ | ++++ | ++++ | ++++ |
| | 2,000 | ++++ | ++++ | ++++ | ++++ | ++++ |
| | 1,000 | ++++ | ++++ | ++++ | ++++ | ++++ |
| | 500 | +++ | ++++ | ++++ | ++++ | ++++ |
| | 250 | +++ | +++ | +++ | +++ | ++++ |
| | 125 | ++ | +++ | +++ | +++ | +++ |

As shown in Table 6, in the results of an inhibitory activity test against the mobility of Enteromorpha spores, the fraction V-e showed the most powerful inhibitory activity at varying concentrations of 250, 500, 1000, 1500, 2000 and 4000 μg/ml, and showed strong inhibitory activity at a concentration of 125 μg/ml(+++). The fraction V-e is fractionated into three fractions by each solvent depending on solubility using organic solvents (hexane, ethyl acetate, methanol). Each of the three fractions was tested for an inhibitory effect against the mobility of Enteromorpha spores, and the results are shown in Table 7 below.

TABLE 7

| | Activity | | |
|---|---|---|---|
| Concentration | V-e | V-e | V-e |
| ((g/ml) | n-hx | EA | MeOH |
| 4,000 | +++ | ++++ | ++++ |
| 2,000 | +++ | ++++ | +++ |
| 1,000 | ++ | ++++ | ++ |
| 500 | ++ | ++++ | ++ |
| 250 | − | ++++ | ++ |
| 125 | − | +++ | ++ |

As shown in Table 7, in the results of an inhibitory activity test against the mobility of Enteromorpha spores, among the fractions V-e fractionated by each solvent depending on solubility, the fraction eluted with the solvent of ethyl acetate showed the most powerful inhibitory activity against the mobility of the spores.

Prep-High Performance Liquid Chromatography (Prep-HPLC)

The fraction obtained by fractionating V-e with the solvent of ethyl acetate was applied to a Prep-HPLC C18 reversed phase column (Microsorb, 21.4×250 mm) and was eluted with a mixed solvent of 80% methanol and 20% water for 60 minutes at a flow rate of 5 ml/min. The absorbance of each of the eluted fractions at 254 nm was measured while collecting eight fractions (k1-k8). Each of the eight fractions was tested for an inhibitory effect against the mobility of Enteromorpha spores, and the results are shown in Table 8 below. The fraction k7 showing the most excellent inhibitory activity against the mobility of the spores in the physiological activity test of the collected eight fractions was used as a sample for analysis of substance structure through gas chromatography/gas mass spectrometer(GC/MS), high-performance liquid chromatography/liquid mass spectrometer(LC/MS), and nuclear magnetic resonance(NMR).

TABLE 8

| Concentration | Activity | | | | | | | |
|---|---|---|---|---|---|---|---|---|
| (μg/ml) | V-e-k1 | V-e-k2 | V-e-k3 | V-e-k4 | V-e-k5 | V-e-k6 | V-e-k7 | V-e-k8 |
| 4,000 | +++ | ++++ | +++ | ++++ | +++ | ++++ | ++++ | ++++ |
| 2,000 | +++ | ++++ | ++ | ++++ | ++++ | ++++ | ++++ | +++ |
| 1,000 | ++ | ++++ | ++ | ++++ | ++++ | ++++ | ++++ | +++ |
| 500 | ++ | +++ | ++ | ++++ | ++++ | +++ | ++++ | +++ |
| 250 | ++ | +++ | ++ | +++ | ++++ | +++ | ++++ | +++ |
| 125 | + | ++ | ++ | +++ | +++ | +++ | ++++ | +++ |

High Performance Liquid Chromatography(HPLC)

The fraction k7 showing the most excellent inhibitory activity against the mobility of the spores in the physiological activity test of physiological activity substances or the collected eight fractions fractionated by Prep-HPLC was applied to a HPLC C18 reversed phase column (Microsorb, 21.4×250 mm, Cosmosil) and was eluted with a mixed solvent of 80% methanol and 20% water for 60 minutes at a flow rate of 1 ml/min. The absorbance of each of the eluted fractions at 254 nm was measured while collecting and analyzing eight fractions.

Gas Chromatography Mass Spectrum

From the HPLC, only the active portion (80% methanol concentration) was recovered, dried and dissolved in methanol, 1 mg of the solution was used in a gas chromatography analysis. In the gas chromatography, a capillary column HP-5(Hewlett-Packard, 30 cm×0.25 mm×0.25 μm) was used and analyzed by split injection (1:50) at an injection rate of 0.6 ml/min using helium as mobile phase gas. The temperature of the injection port of the column was 230° C. and the initial temperature of the column was 100° C. The column was kept at a temperature of 100° C. for 2 minutes, and then was raised to 150° C. at a rate of 4° C./min. Thereafter, the column was raised to 200° C. at a rate of 3° C./min and subsequently was raised to 250° C. at a rate of 7° C./min, at which temperature the column was kept. In this regard, 70 eV was used at 280° C. in the electron ionization mode of an ion source (see Table 9).

TABLE 9

| Parameter | Condition |
|---|---|
| Column | HP-5 column |
| | (30 cm × 0.25 mm × 0.25 μm) |
| Injector temperature | 230° C. |
| Detector | 230° C. |
| Initial temperature | 100° C. |
| Programming rate | 4° C./min to 150° C. |
| | 3° C./min to 200° C. |
| | 7° C./min to 250° C. |
| Final temperature | 250° C. for 6 min |
| Flow rate of mobile phase gas | 0.6 ml/min |
| Split ratio | 1:50 |

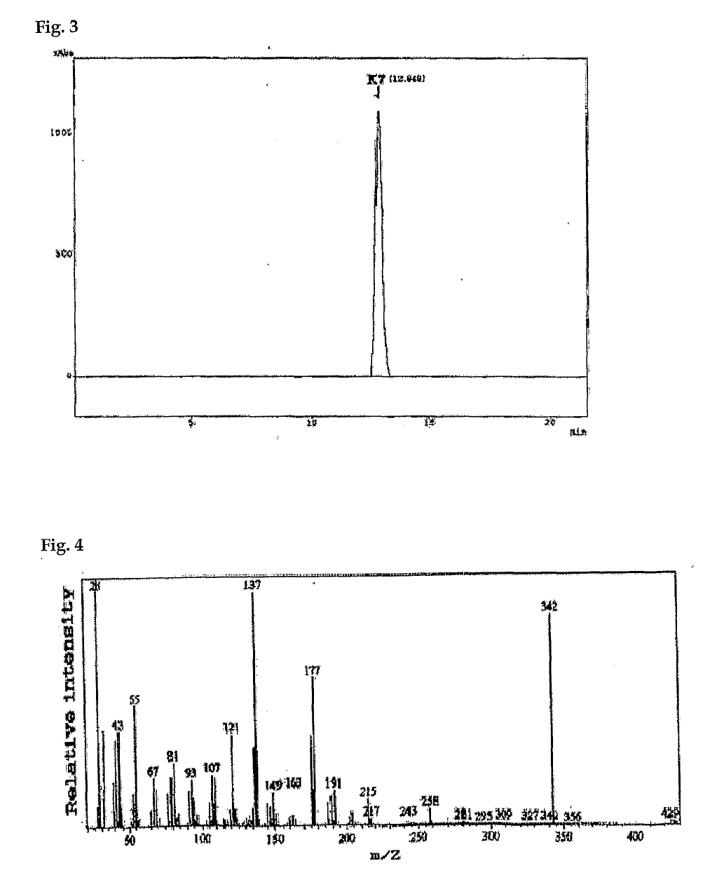

The molecular weight of the fraction V-e-K7 was determined by LC/GC MS (mass spectrum) (see FIGS. 3 and 4), and the result of GC-MS for the fraction V-e-K7 is as follows. Rt: 71.56 min; Molecular ion: M+ −342.

### EXAMPLE 3

### Solvent Fractionation of Sargassum sp. Extract

In order to investigate inhibitory substances against the mobility of Enteromorpha spores from extracts of specific Sargassum sp., i.e., Sargassum sp. (139), Sargassum sp. (365) and Sargassum sp. (383), organic solvent fractions were made.

Marine algae were extracted with each of hexane, methyl alcohol and water. The hexane and methyl alcohol extracts were filtered and the filtrates were concentrated with a vacuum concentrator at 30° C. The water extract was freeze-dried with a freeze dryer.

Each of the extracts was tested for physiological activity, and the results are shown in Table 10 below. In the results of an inhibitory activity test against the mobility of Enteromorpha spores, the hexane extract showed powerful inhibitory effect (inhibition of more than 95%: ++++) at 10,000 μg/ml, 7,500 μg/ml and 5,000 μg/ml, strong activity (95-75%: +++) at 2,500 μg/ml, and moderate activity (75-50%: ++) at 1,250 μg/ml.

The methyl alcohol extract showed strong activity (+++) at 10,000 μg/ml and 75,000 μg/ml, but no activity at 1,250 μg/ml. Also, the water extract showed moderate activity (++) at 10,000 μg/ml and 7,500 (g/ml, weak activity at 5,000 (g/ml, and no activity at 2,500 (g/ml and 1,250 (g/ml.

As a result, the hexane extract showed an excellent effect, and thus, used as a sample for the isolation and purification of antifouling components.

TABLE 10

| Concentration | Activity | | |
|---|---|---|---|
| ((g/ml) | n-hexane | methyl alcohol | water |
| 10,000 | ++++ | +++ | ++ |
| 7,500 | ++++ | +++ | ++ |
| 5,000 | ++++ | ++ | + |
| 2500 | +++ | + | − |
| 1250 | ++ | − | − |

First Silica Gel Column Chromatography

A column was packed with silica gel (70-230 meshes) in hexane. The hexane extract sample was applied to the column, and the column was eluted sequentially with hexane (10), hexane:methylene chloride (7.5:2.5), hexane:methylene chloride (5:5), hexane:methylene chloride (2.5:7.5), methylene chloride (10), methylene chloride:ethylene acetate (8:2), methylene chloride:ethyl acetate (6:4), methylene chloride:ethyl acetate (4:6), methylene chloride:ethyl acetate (2:8), ethyl acetate (10), ethyl acetate:methyl alcohol (9:1), ethyl acetate:methyl alcohol (8:2), ethyl acetate:methyl alcohol (7:3), ethyl acetate:methyl alcohol (5:5), and methyl alcohol (10). These extraction solvents were passed through the column in a volume of 100 ml for the extraction solvents from hexane to methylene chloride, and in a volume of 300 ml for the extraction solvents from methylene chloride (8:2) to methyl alcohol, at a flow rate of 3 ml/min (see Table 11).

TABLE 11

| Extraction solvents | Volume (ml) |
|---|---|
| Hexane (10) | 100 |
| Hexane:methylene chloride | 100 |
| (7.5:2.5) | |
| Hexane:methylene chloride (5:5) | 100 |
| Hexane:methylene chloride | 100 |
| (2.5:7.5) | |
| Methylene chloride (10) | 100 |
| Methylene chloride:ethyl | 300 |
| acetate (8:2) | |
| Methylene chloride:ethyl | 300 |
| acetate (6:4) | |
| Methylene chloride:ethyl | 300 |
| acetate (4:6) | |
| Methylene chloride:ethyl | 300 |
| acetate (2:8) | |
| Ethyl acetate (10) | 300 |
| Ethyl acetate:methyl alcohol | 300 |
| (9:1) | |
| Ethyl acetate:methyl alcohol | 300 |
| (8:2) | |
| Ethyl acetate:methyl alcohol | 300 |
| (7:3) | |
| Ethyl acetate:methyl alcohol | 300 |
| (6:4) | |
| Ethyl acetate:methyl alcohol | 300 |
| (5:5) | |
| Methyl alcohol (10) | 300 |

The elutes were fractionated into six fractions by performing TLC in a mixed solvent of hexane:methylene chloride:ethyl acetate (3:1:1). The six fractions (I-VI) were tested for physiological activity, and the results are shown in Table 12 below.

TABLE 12

| Concentration | Activity | | | | | |
|---|---|---|---|---|---|---|
| ((g/ml) | I | II | III | IV | V | VI |
| 4,000 | ++ | ++++ | +++ | +++ | ++++ | +++ |
| 2,000 | ++ | ++ | +++ | +++ | ++++ | ++ |
| 1,500 | ++ | + | ++ | +++ | ++++ | ++ |
| 1,000 | + | + | ++ | ++ | ++++ | ++ |
| 750 | − | + | ++ | ++ | +++ | ++ |
| 250 | − | + | + | + | +++ | + |

At a concentration of 4,000 (g/ml, the fraction V showed powerful inhibitory activity (++++) against the mobility of the spores, and the fractions II, III, IV and VI showed strong activity (+++). At 2,000 (g/ml and 1,500 (g/ml, the fraction V showed powerful activity (++++), and at 750 (g/ml and 250 (g/ml, it showed a reduction in activity but maintained strong activity (+++). As a result, the fraction V within the range of methylene chloride:ethyl acetate (2:8), which shows the most excellent activity among the fractions I-VI, was used as a sample for the isolation and purification of antifouling components by the second silica gel column chromatography.

Second Silica Gel Column Chromatography

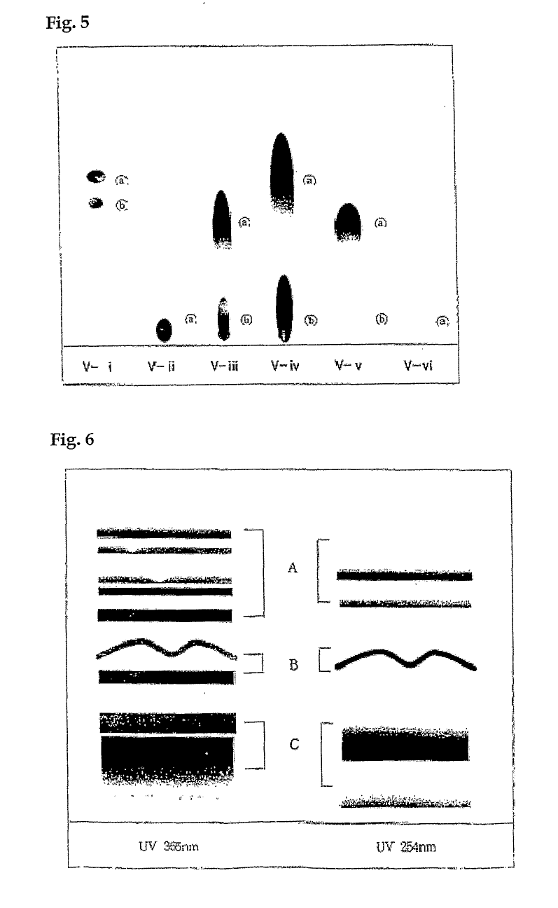

A column was packed with silica gel (70-230 meshes) in hexane. The fraction V as a sample was applied to the column, and the column was eluted sequentially with hexane (10), hexane:methylene chloride (9:1), hexane:methylene chloride (8:2), hexane:methylene chloride (7:3), hexane:methylene chloride (6:4), hexane:methylene chloride (5:5), hexane:methylene chloride (4:6), hexane:methylene chloride (3:7), hexane:methylene chloride (2:8), hexane:methylene chloride (1:9), methylene chloride (10), methylene chloride:ethyl acetate (9:1), methylene chloride:ethyl acetate (8:2), methylene chloride:ethyl acetate (7:3), methylene chloride:ethyl acetate (6:4), methylene chloride:ethyl acetate (5:5), methylene chloride:ethyl acetate (4:6), methylene chloride:ethyl acetate (3:7), methylene chloride:ethyl acetate (2:8), methylene chloride:ethyl acetate (1:9), ethyl acetate (10), ethyl acetate:methyl alcohol (9:1), ethyl acetate:methyl alcohol (8:2), ethyl acetate:methyl alcohol (7:3), ethyl acetate:methyl alcohol (6:4), ethyl acetate:methyl alcohol (5:5), and methyl alcohol (10). Each of these extraction solvents was passed through the column in a volume of 100 ml at a flow rate of 3 ml/min (see Table 13). Then, each of eluates was subjected to TLC in a mixed solvent of hexane:methylene chloride:ethyl acetate (3:1:1) so as to make six fractions (V-i to V-vi). The TLC diagram for each of the fractions is shown in FIG. 5, the Rf values for the respective fractions are shown in Table 14. The six fractions were tested for inhibitory activity against the mobility of spores, and the results are shown in Table 15 below.

TABLE 13

| Extraction solvent | Volume (ml) |
|---|---|
| Hexane (10) | 100 |
| Hexane:methylene chloride (9:1) | 100 |
| Hexane:methylene chloride (8:2) | 100 |
| Hexane:methylene chloride (7:3) | 100 |
| Hexane:methylene chloride (6:4) | 100 |
| Hexane:methylene chloride (5:5) | 100 |
| Hexane:methylene chloride (4:6) | 100 |
| Hexane:methylene chloride (3:7) | 100 |
| Hexane:methylene chloride (2:8) | 100 |
| Hexane:methylene chloride (1:9) | 100 |
| Methylene chloride (10) | 100 |
| Methylene chloride:ethyl acetate (9:1) | 100 |
| Methylene chloride:ethyl acetate (8:2) | 100 |
| Methylene chloride:ethyl acetate (7:3) | 100 |
| Methylene chloride:ethyl acetate (6:4) | 100 |
| Methylene chloride:ethyl acetate (5:5) | 100 |
| Methylene chloride:ethyl acetate (4:6) | 100 |
| Methylene chloride:ethyl acetate (3:7) | 100 |
| Methylene chloride:ethyl acetate (2:8) | 100 |
| Methylene chloride:ethyl acetate (1:9) | 100 |
| Ethyl acetate (10) | 100 |
| Ethyl acetate:methyl alcohol (9:1) | 100 |
| Ethyl acetate:methyl alcohol (8:2) | 100 |
| Ethyl acetate:methyl alcohol (7:3) | 100 |
| Ethyl acetate:methyl alcohol (6:4) | 100 |
| Ethyl acetate:methyl alcohol (5:5) | 100 |
| Methyl alcohol (10) | 100 |

TABLE 14

| | Rf((100)in |
|---|---|
| | n-hexane-methylene chloride-ethyl acetate (3:1:1) |
| V-i - \textcircled{a} | 47 |
| V-i - \textcircled{b} | 37 |
| V-ii - \textcircled{a} | 9 |
| V-ii - \textcircled{b} | 0 |
| V-iii - \textcircled{a} | 41 |
| V-iii - \textcircled{b} | 10 |
| V-iv - \textcircled{a} | 56 |
| V-iv - \textcircled{b} | 20 |
| V-v - \textcircled{a} | 37 |
| V-v - \textcircled{b} | 10 |
| V-vi - \textcircled{a} | 8 |
| V-vi - \textcircled{b} | 0 |

TABLE 15

| Concentration | Activity | | | | | |
|---|---|---|---|---|---|---|
| (μg/ml) | V-i | V-ii | V-iii | V-iv | V-v | V-vi |
| 4,000 | ++ | ++++ | ++++ | ++++ | ++++ | ++ |
| 2,000 | ++ | +++ | ++++ | ++++ | ++++ | ++ |
| 1,500 | ++ | ++ | ++++ | ++++ | +++ | ++ |
| 1,000 | + | ++ | ++++ | +++ | +++ | + |
| 750 | + | ++ | ++++ | +++ | +++ | + |
| 500 | + | + | ++++ | ++ | ++ | − |
| 375 | + | + | +++ | ++ | ++ | − |
| 250 | − | + | +++ | + | + | − |

As a result, the fraction V-iii showed the most excellent activity, and thus, was used as a sample for the isolation and purification of antifouling components by Prep-TLC.

The fraction V-iii showing the most excellent activity in the physiological activity test of the fractions obtained by performing the concentration gradient of the organic solvents by the second silica gel column chromatography was developed on Prep-TLC with the use of a mixed solvent of hexane:methylene chloride:ethyl acetate (3:1:1) to obtain three fractions, i.e., V-iii-a, V-iii-b and V-iii-c.

The TLC diagram for each of the fractions is shown in FIG. 6, the Rf values for the respective fractions are shown in Table 16. The inhibitory effect of the three fractions against the mobility of Enteromorpha spores is shown in Table 17 below.

TABLE 16

| | Rf (×100) in |
|---|---|
| | n-hexane-methylene chloride-ethyl acetate (3:1:1) |
| V-iii-a | 37-42 |
| V-iii-b | 21-37 |
| V-iii-c | 0-21 |

TABLE 17

| Concentration | Activity | | |
|---|---|---|---|
| (μg/ml) | V-iii-a | V-iii-b | V-iii-c |
| 4,000 | ++++ | +++ | ++++ |
| 2,000 | +++ | +++ | +++ |
| 1,500 | +++ | ++ | +++ |
| 1,000 | +++ | ++ | ++ |
| 750 | +++ | ++ | ++ |
| 500 | +++ | ++ | ++ |

As a result, the fraction V-iii-a maintained activity despite an increase in dilution rate, and thus, was used as a sample for the isolation and purification of antifouling components by Prep-HPLC.

Prep-HPLC

The active fraction V-iii-a obtained in Prep-TLC was applied to a Prep-HPLC C18 reversed phase column (Microsorb, 21.4×250 mm). The mobile phase was eluted with a mixed solvent of 60% acetonitrile and 40% water for 120 minutes at a flow rate of 20 ml/min. The absorbance of the eluted fraction at 213 nm was measured while collecting six fractions (K1-K6) showing the peak absorbance.

The chromatogram obtained by applying the active fraction V-iii-a obtained from Prep-TLC to Prep-TLC and analysis HPLC is shown in FIG. 7, and each of the fractions (K1-K6) collected from the Prep-HPLC of the active fraction V-iii-a obtained from Prep-TLC was tested for the inhibitory activity against the mobility of Enteromorpha spores, and the results are shown in Table 18 below.

TABLE 18

| | Activity | | | | | |
|---|---|---|---|---|---|---|
| Concentration ((g/ml) | K1 | K2 | K3 | K4 | K5 | K6 |
| 4,000 | | +++ | ++++ | ++++ | ++++ | ++++ |
| 2,000 | ++ | +++ | ++++ | ++++ | ++++ | ++++ |
| 1,500 | ++ | ++ | ++++ | ++++ | ++++ | +++ |
| 1,000 | + | ++ | ++++ | ++++ | ++++ | +++ |
| 500 | + | ++ | +++ | ++++ | +++ | ++ |
| 250 | ( | + | +++ | ++++ | ++ | + |
| 125 | ( | + | ++ | ++++ | ++ | + |
| 62.5 | ( | ( | ++ | ++++ | + | + |

As a result, the fraction K4 was the most excellent in an inhibitory effect against the mobility of Enteromorpha spores among the six collected fractions (K1-K6).

### EXAMPLE 3

### Identification of Sargassum confusum-Derived Antifouling Substances

The fractions A-H in Example 2 were subjected to NMR to identify the antifouling substances.

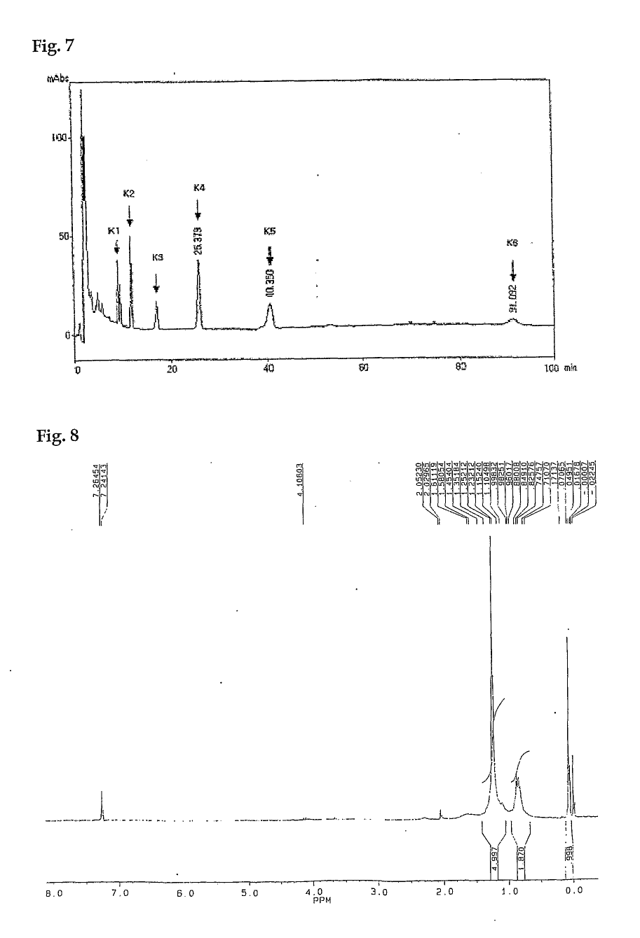

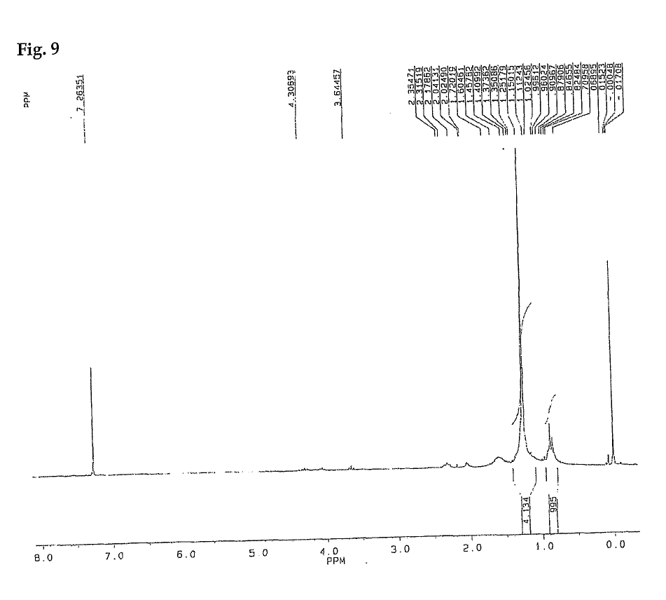

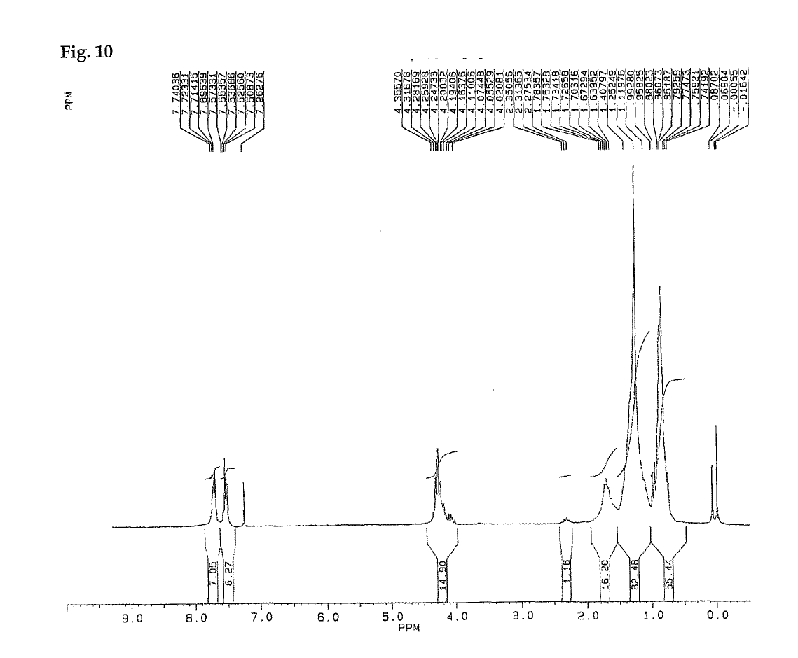

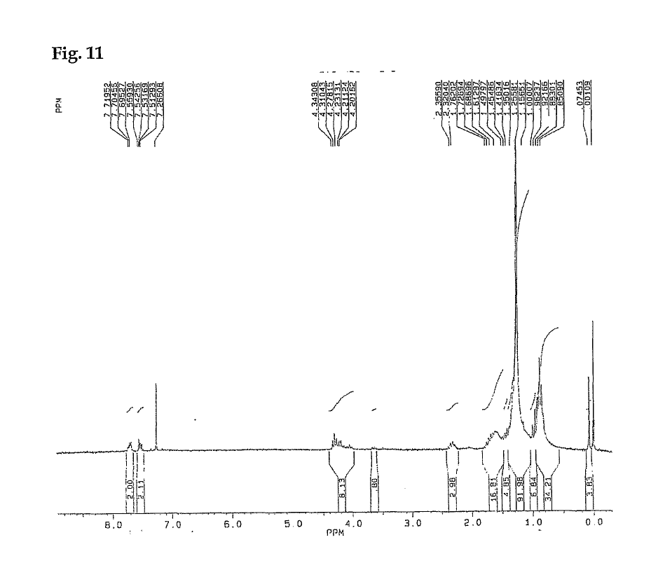

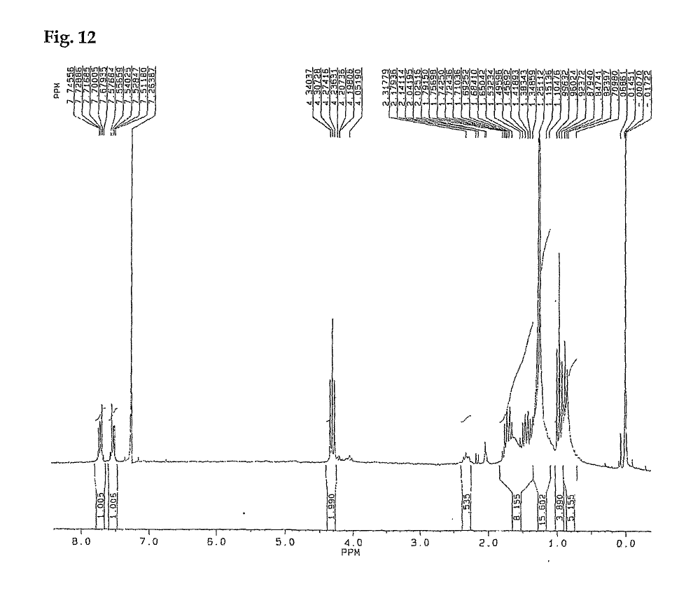

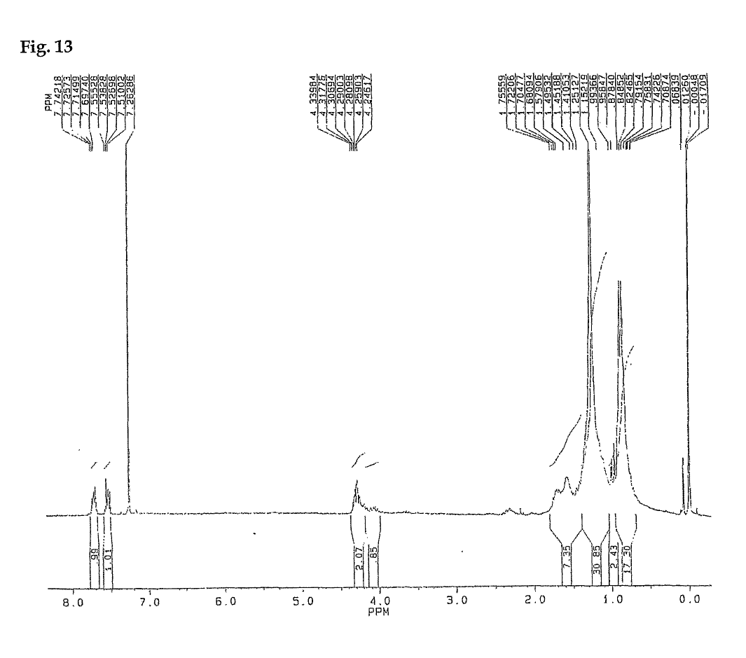

As a result of analysis of the fractions A-H by hydrogen NMR, it was found that the fraction A was in the form of a combination of hexadecane and octadecane, the fraction B was eicosane, the fraction B was 1-pentadecanol, the fraction C was 1-eicosanol, the fraction D was 1-pentadecanol, the fraction E was dibutyl phthalate, the fraction F was dioctyl phthalate, the fraction G was diisononyl phthalate, and the fraction H was dicyclohexyl phthalate, respectively (see FIGS. 8 to 14). The above fractions have the structures of the following formulae:

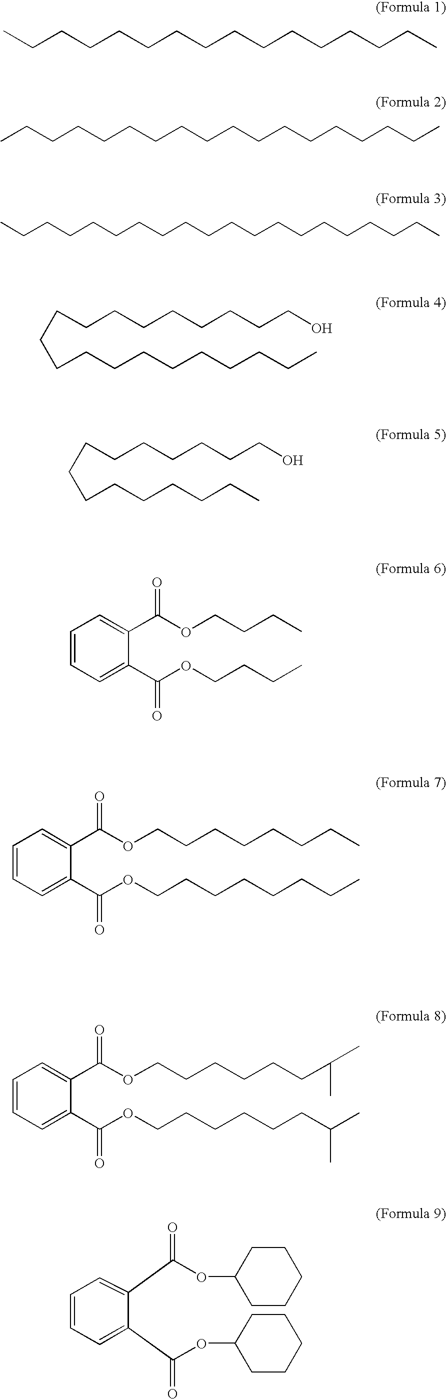

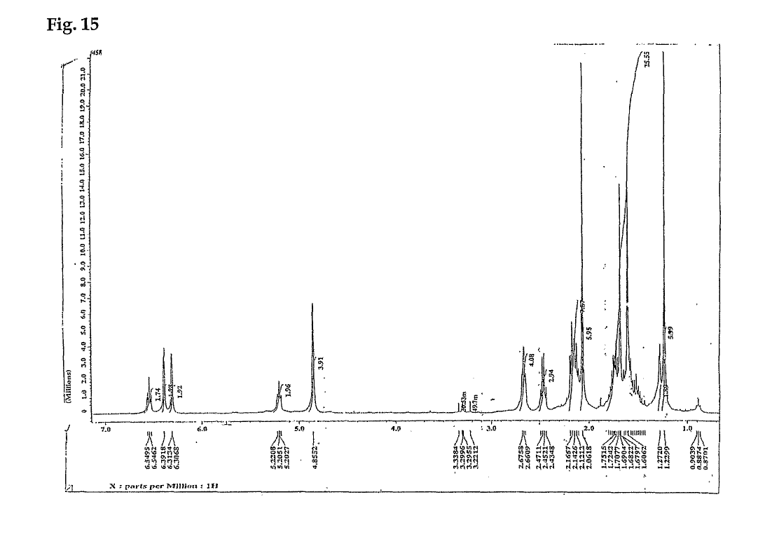

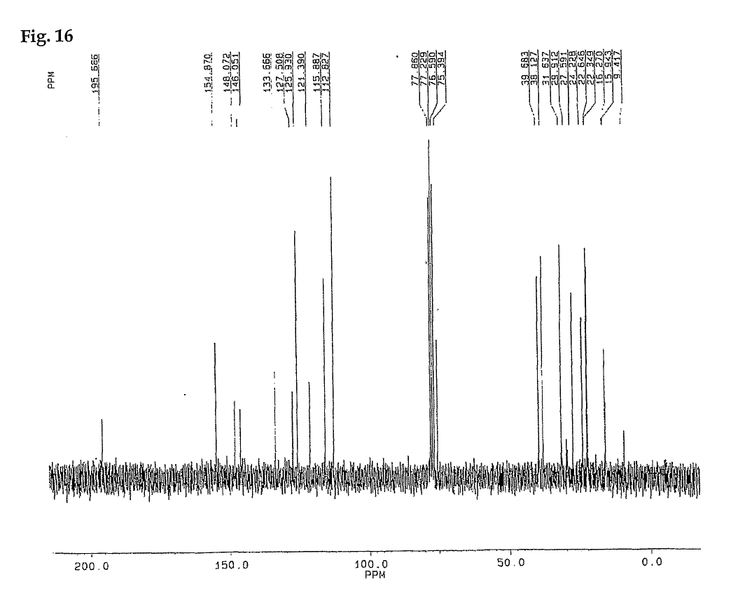

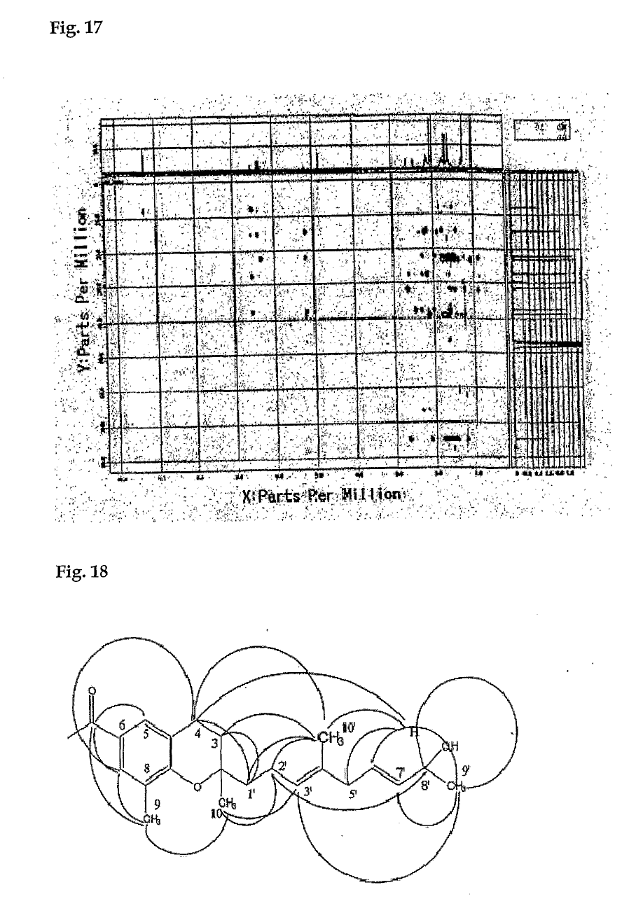

In addition, hydrogen nuclear magnetic resonance (¹H NMR) spectrum of the fraction V-e-k7 in Example 3 was obtained from the Bruker AC-200 spectrometer (500 MHz), and carbon nuclear magnetic resonance (¹³C NMR) spectrum was obtained from the Bruker AC-800 spectrometer (800 MHz) (see FIGS. 15 to 18 and Table 19).

TABLE 19

| Position | (C(CDCl3) | (H(CDCl3) |
|---|---|---|
| 2 | 39.68 | — |
| 3 | 31.63 | 2.64 (q.) |
| 4 | 27.59 | 2.66 |
| 4a | 112.83 | — |
| 5 | 146.05 | 6.52 (dd) |
| 6 | 133.67 | — |
| 7 | 148.07 | 6.52 (dd) |
| 8 | 125.93 | — |
| 8a | 154.07 | — |
| 9 | 22.35 | 2.06 (s) |
| 10 | 22.65 | 1.67 |
| 1′ | 38.12 | 1.25 (dr, s) |
| 2′ | 24.30 | 2.14 (m) |
| 3′ | 127.54 | 5.20 (t) |
| 4′ | 121.39 | — |
| 5′ | 24.30 | 2.14 (m) |
| 6′ | 115.84 | 6.30 (d) |
| 7′ | 117.64 | 6.39 (d) |
| 8′ | 75.40 | 3.35 (d) |
| 9′ | 15.94 | 2.06 (s) |
| 10′ | 16.27 | 1.63 (s) |
| —OH | — | 4.80 (br, s) |
| —CHO | 195.68 | 9.36 (s) |

¹H NMR (500 MHz, CDCl₃/TMS) spectrum showed five skeletons in which two meta pairs constitute another pair at one side where δ6.52 connected with aromatic protons is formed in pair, three pairs of 5.21 is formed at both sides where two olefin protons δ6.35 pair is formed, and four methyl protons constitute δ1.63, 1.67 and 2.06 as each single body. One among four molecules is δ2.46, and benzo pyran nucleus containing two hydrogen atoms forms three pairs of (2.66 at allylic position and acts.

Also, (9.30 as a single body indicates aldehyde proton, (4.80 denotes extensive positions of hydroxyl protons and is exchanged with D2O. (1.25 and 2.14 indicates extensive positions of methylenic protons and their data denotes 14, 15, i.e., a part of the prenyl side-chain double bond as a mixture and dehydro benzo pyran.

13C NMR (800 MHz, CDCl3/TMS) spectrum indicates twenty-two carbon signals. The signal of (195.68 indicates aldehyde, olefinic carbons are represented by (127.5, 121.3, 117.6 and 115.8, and (148.05 and 146.07 as single bodies denote aldehyde comprised of aromatic nucleus and two meta pairs. This could not suggest that aldehyde is a side chain, but it could be found that aldehyde is aromatic nucleus and a fragment of mass m/e 121.

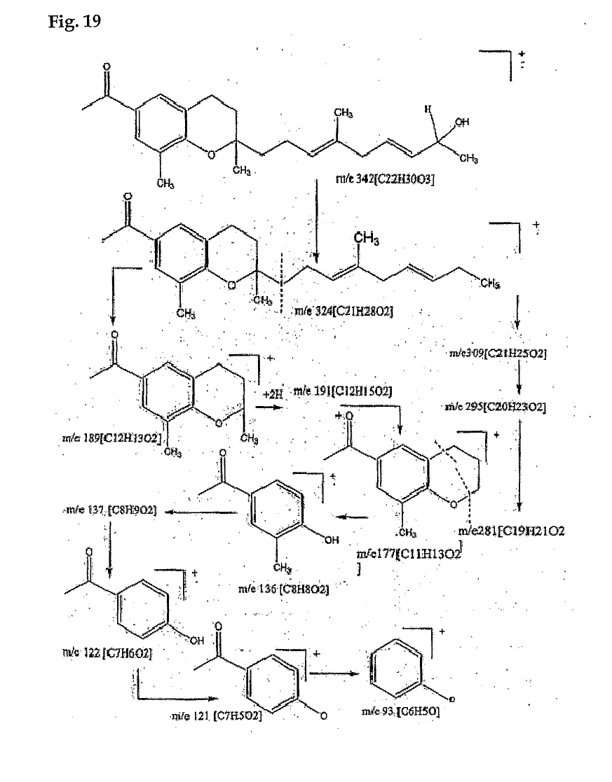

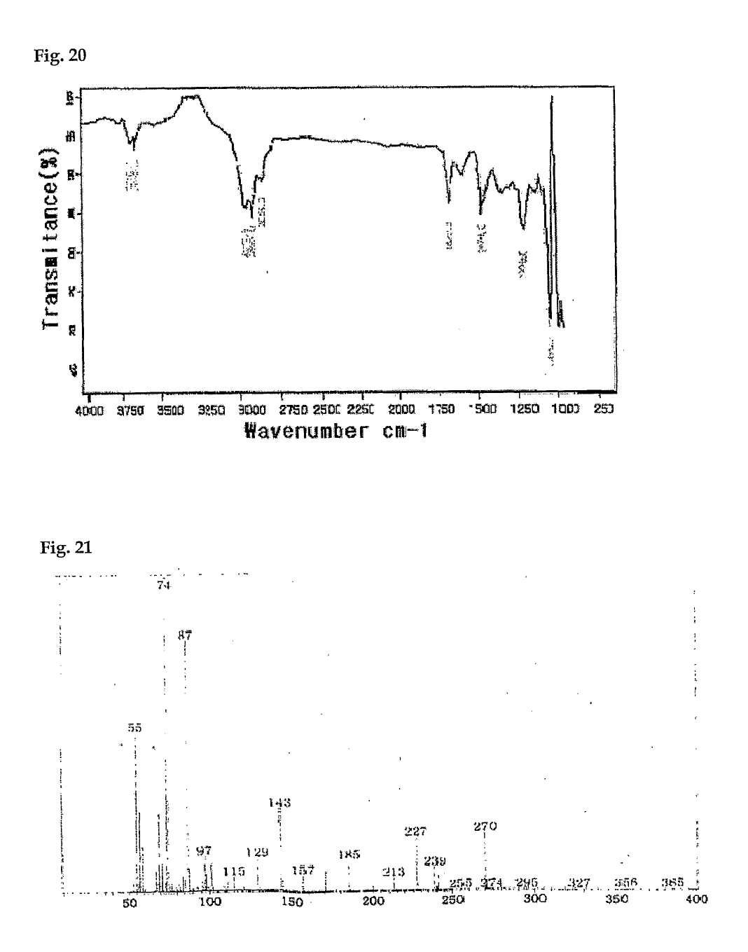

As a result of analysis of the above data, it was found that it is 2-(3′,6′-dienyl-8′-hydroxy-4′-methylnonane)-2,8-dimethyl-2H-chroman-6-ol”, and a partial pattern by mass was obtained (see FIG. 19).

In addition, the fraction V-e-K7 was measured by using the infrared absorption spectrum (Fourier Infrared Spectrophotometer, Mattson, USA) KBr pellet method (CHO, Chong-Soo, et al., 1987).

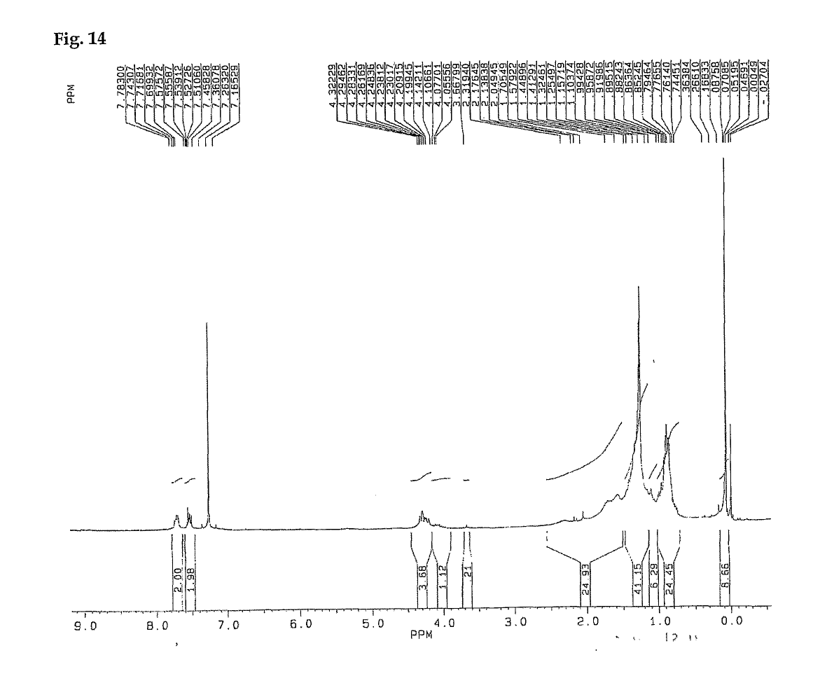

As a result of analysis of the infrared spectrum for the fraction V-e-K7 (see FIG. 14), v max (KBr)-3710, 3669(—OH), 2972, 2933, 2865(CH3, St), 1680(═C=0, St), 1597, 1520 and 1474 cm-1 (aromatic function) are obtained. The collected data are synthesized and analyzed, and as a result, antifouling substances separated from Sargassum confusum has a structure of the following Formula 10:

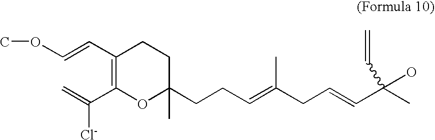

### EXAMPLE 8

### Identification of Sargassum sp.-derived Antifouling Substances

The fraction K4 in Example 4 was used as a sample for analysis of a substance structure using GC-MS, LC-MS and NMR.

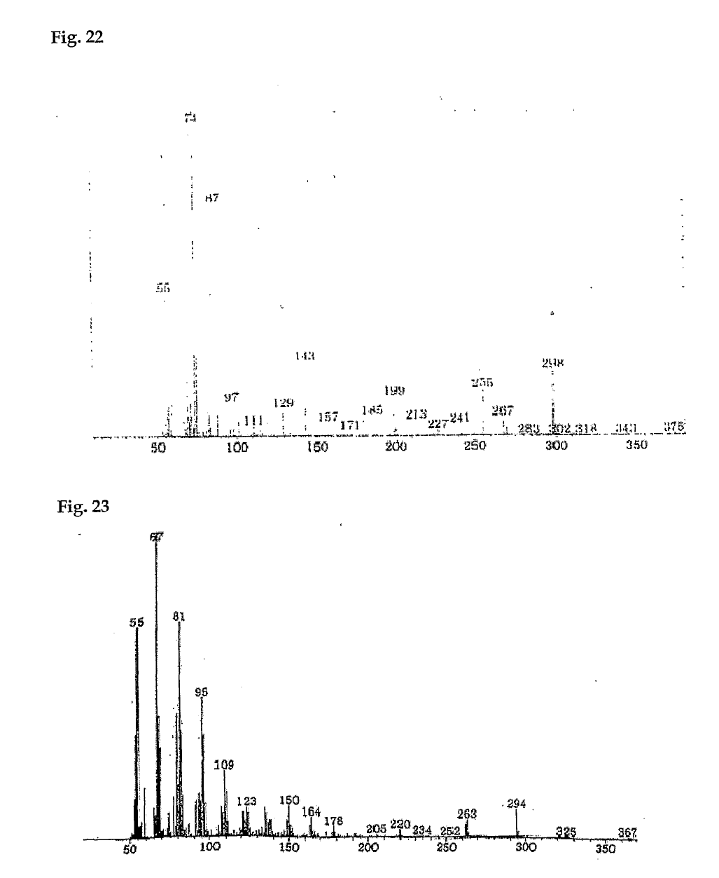

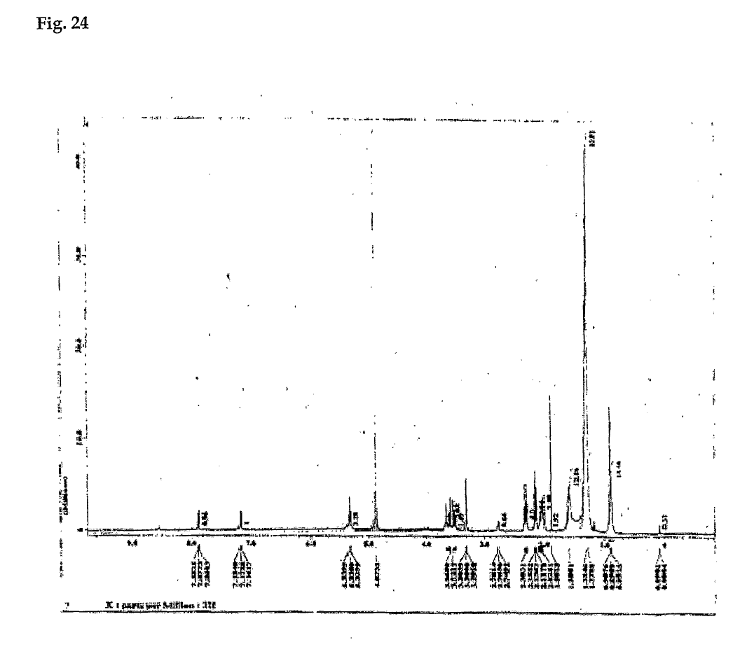

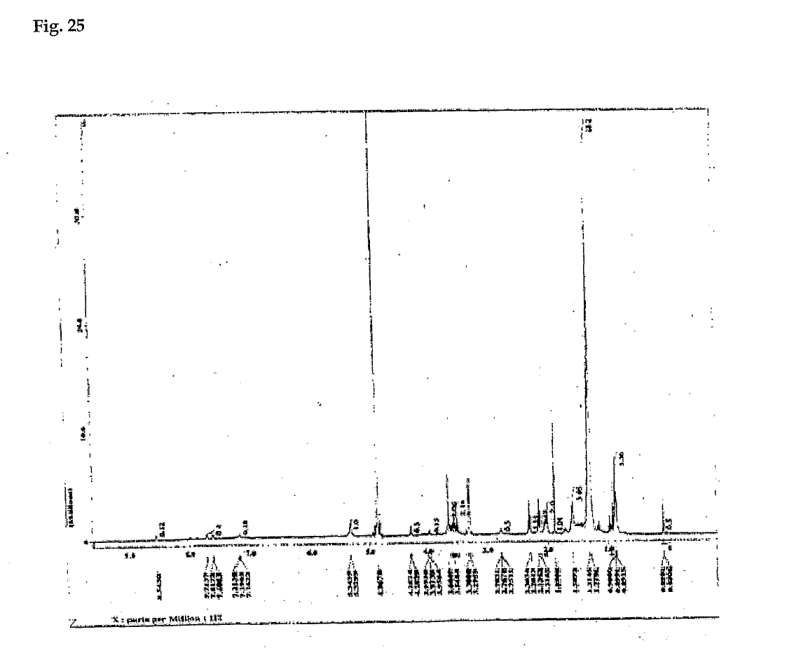

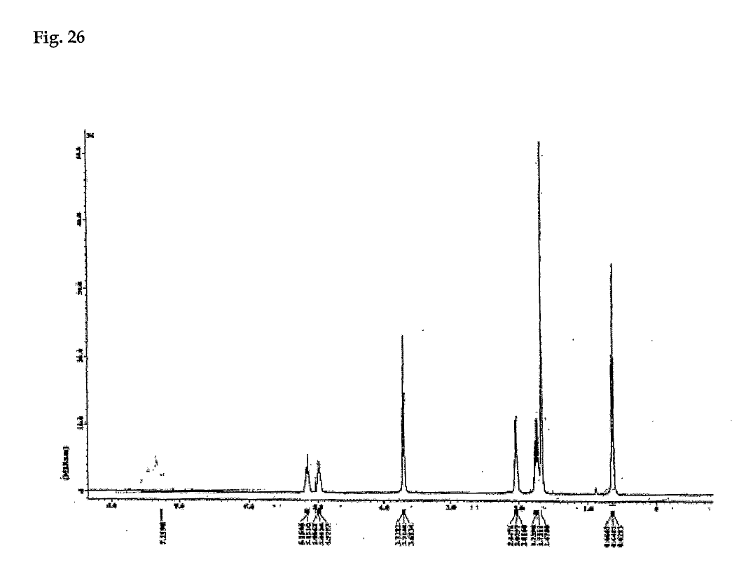

GC-MS results for the fraction K4 are shown in FIGS. 21 to 23, and the NMR data for the fraction K4 are shown in FIGS. 24 to 26. Also, the fraction K4 has the structures of the following Formulae 11 to 13:

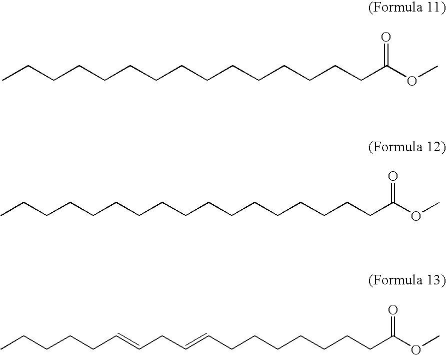

Resultantly, it was found that the novel antifouling substances with excellent antifouling activity, isolated and purified from Sargassum sp. (139, 365, 383) were methyl palmitate, methyl stearate and methyl linoleate, which are fatty acids.

### EXAMPLE 7

### Safety Test

Each of hexadecane, octadecane, eicosane, 1-eicosanol, 1-pentadecanol, dibutyl phthalate, dioctyl phthalate, diisononyl phthalate, dicyclohexyl phthalate, and 6-formyl-3,4-dehydro-2,8-dimethyl-2-(3′,6′-dienyl-8′-hydroxy-4′-methylnonane)-2H-1-benzopyrane is dissolved in dimethylsulfoxide (DMSO) and diluted with water. Then, 10 mg/kg of each of the dilutions was administered to each mouse group (consisting of 10 mice), and the mice were observed for 7 days. The observation result showed no death of the mice.

The physical properties of methyl palmitate are as follows: a melting point of 32-34° C., a boiling point of 163-164° C., and a density of 0.852. This compound was administered orally to mice and tested for toxicity, and the results were as follows: LD₅₀>20 mg/kg. When a stimulus was applied to eyes of rabbits, only a transient stimulus phenomenon occurred. Also, similarly, when a stimulus was applied to transdermal routes of the human body, only a very mild stimulus remained. Blugil fish which is a kind of trout was tested for toxicity against water, the result was as follows: LC₅₀=1000 ml/l.

Methyl stearate is an opaque white-colored crystal and has the following physical properties: a melting point of 40-42° C., and a boiling point of 181-182° C. The compound may be harmful by suction, intake, epidermal absorption, but gives no stimulus. That is, the following test result was obtained: LD50>17.2 g/kg.

Methyl linoleate is a colorless liquid and has the following physical properties: a melting point of −35° C., a boiling point of 192° C. and a density of 0.889.

### EXAMPLES 8-24

### Preparation of Antifouling Paints

Resin and rosin were completely dissolved in xylene and a small amount of methyl isobutyl ketone. To the solution, zinc oxide and iron oxide as pigments were added, and dispersed two times with a sand mill. To this mixture, antifouling substances and thickener given in Table 20 below were added. The resulting mixture was stirred with a high-speed stirrer at 3500 rpm for 60 minutes. Then, the remaining ketone solvent was added and stirred, thus preparing antifouling paints.

TABLE 20

| | Antifouling | Resin | Solvent | | |
|---|---|---|---|---|---|
| | substance (5 wt | (10 wt | (23 wt | Pigment (40 wt | Thickener (2 wt |
| | parts) | parts) | parts) | parts) | parts) |
| Example 8 | hexadecane | 5 wt | 10 wt | 25 wt parts | Polyimide wax |
| | | parts | parts of | of zinc | |
| | | of | xylene/13 wt | oxide/15 wt | |
| | | vinyl | parts | parts of | |
| | | resin/5 wt | of methyl | iron oxide | |
| | | parts | isobutyl | | |
| | | of | ketone | | |
| | | rosin | | | |
| Example9 | octadecane | The | The same | The same as | The same as |
| | | same as | as above | above | above |
| | | above | | | |
| Example | eicosane | The | The same | The same as | The same as |
| 10 | | same as | as above | above | above |
| | | above | | | |
| Example | 1-eicosanol | The | The same | The same as | The same as |
| 11 | | same as | as above | above | above |
| | | above | | | |
| Example | 1-pentadecanol | The | The same | The same as | The same as |
| 12 | | same as | as above | above | above |
| | | above | | | |
| Example | dibutyl | The | The same | The same as | The same as |
| 13 | phthalate | same as | as above | above | above |
| | | above | | | |
| Example | dioctyl | The | The same | The same as | The same as |
| 14 | phthalate | same as | as above | above | above |
| | | above | | | |
| Example | diisononyl | The | The same | The same as | The same as |
| 15 | phthalate | same as | as above | above | above |
| | | above | | | |
| Example | dicyclohexyl | The | The same | The same as | The same as |
| 16 | phthalate | same as | as above | above | above |
| | | above | | | |
| Example | methyl | The | The same | The same as | The same as |
| 17 | palmitate | same as | as above | above | above |
| | | above | | | |
| Example | Methyl | The | The same | The same as | The same as |
| 18 | stearate | same as | as above | above | above |
| | | above | | | |
| Example | Methyl | The | The same | The same as | The same as |
| 19 | linoleate | same as | as above | above | above |
| | | above | | | |
| Example | 6-formyl-3,4- | The | The same | The same as | The same as |
| 20 | dehydro-2,8- | same as | as above | above | above |
| | dimethyl-2- | above | | | |
| | (3′,6′-dienyl- | | | | |
| | 8′-hydroxy-4′- | | | | |
| | methylnonane)- | | | | |
| | 2H-1- | | | | |
| | benzopyrane | | | | |
| Example | 6-formyl-3,4- | 5 wt | The same | The same as | The same as |
| 21 | dehydro-2,8- | parts | as above | above | above |
| | dimethyl-2- | of | | | |
| | (3′,6′-dienyl- | acryl | | | |
| | 8′-hydroxy-4′- | resin/5 wt | | | |
| | methylnonane)- | parts | | | |
| | 2H-1- | of | | | |
| | benzopyrane | rosin | | | |
| Example | octadecane | The | The same | The same as | The same as |
| 22 | | same as | as above | above | above |
| | | above | | | |
| Example | 1-eicosanol | The | The same | The same as | The same as |
| 23 | | same as | as above | above | above |
| | | above | | | |
| Example | dicyclohexyl | The | The same | The same as | The same as |
| 24 | phthalate | same as | as above | above | above |
| | | above | | | |

### EXAMPLE 25

### Preparation of Booster-Containing Paint

4 parts by weight of Zinc pyrithione as a booster was added to a mixture of the same composition as in Example 13, thus preparing an antifouling paint. The addition of the booster was performed together with the pigment before the dispersion step.

### EXAMPLE 26

### Preparation of Paint Containing Mixture of Antifouling Substances

An antifouling paint was prepared in the same manner as in Example 21 except that dioctyl phthalate was substituted for half of the antifouling substance.

### COMPARATIVE EXAMPLE 1

An antifouling paint was prepared in the same manner as in Example 13 except that the antifouling substance was used in an amount of 1 wt part.

### COMPARATIVE EXAMPLE 2

An antifouling paint was prepared in the same manner as in Example 13 except that the antifouling substance was used in an amount of 1 wt part.

### COMPARATIVE EXAMPLE 3

An antifouling paint was prepared in the same manner as in Example 19 except that the antifouling substance was used in an amount of 1 wt part.

### TEST EXAMPLE 1

Three samples for each antifouling paint, prepared by treating KSD 3501 rolled steel sheets (300×300×3.2 mm) according to the KSM 5569 method, were coated with tar/vinyl resin for rust prevention. Then, each of the samples was spray-coated with each of the antifouling paints prepared in Examples 5-26 and Comparative Examples 1-3 to a dry thickness of 150 μm.

The coated panels were dried at 75% RH and 25° C. for 1 week, and then, immersed in the marine area with a water depth of 2 m in the Korean east sea. After 12 months, the panels were observed. The arithmetic mean of the fouling areas of the three samples was calculated for an effective area of 52,000 mm² defined by a line at a distance of 70 mm downward from the upper edge of the samples, a line at a distance of 30 mm upward from the lower edge, and a line at a distance of 20 mm inward from each of both side edges. The calculated arithmetic mean was expressed in a unit of 5%, and the results are shown in Table 21 below.

TABLE 26

| | Fouling area (%) | | |
|---|---|---|---|
| | Slime | Algae | Barnacle |
| Example 8 | 0 | 0 | 0 |
| Example 9 | 5 | 0 | 0 |
| Example 10 | 15 | 5 | 0 |
| Example 11 | 0 | 0 | 0 |
| Example 12 | 15 | 5 | 0 |
| Example 13 | 0 | 0 | 0 |
| Example 14 | 5 | 0 | 0 |
| Example 15 | 5 | 0 | 0 |
| Example 16 | 0 | 0 | 0 |
| Example 17 | 0 | 0 | 0 |
| Example 18 | 0 | 0 | 0 |
| Example 19 | 0 | 0 | 0 |
| Example 20 | 0 | 0 | 0 |
| Example 21 | 5 | 0 | 0 |
| Example 22 | 5 | 0 | 0 |
| Example 23 | 10 | 5 | 0 |
| Example 24 | 5 | 0 | 0 |
| Example 25 | 10 | 5 | 0 |
| Example 26 | 5 | 0 | 0 |
| Comparative | 100 | 100 | 50 |
| Example 1 | | | |
| Comparative | 100 | 100 | 60 |
| Example 1 | | | |
| Comparative | 100 | 100 | 65 |
| Example 1 | | | |

As can be seen from the above results, the inventive antifouling paints have equal or higher antifouling performance than that of the existing antifouling paints containing organic tin compounds.

### TEST EXAMPLE 2

A liquid medium obtained by diluting PAGS by two-fold serial dilution was placed on a 96-multiwell plate and inoculated with 10⁴ cfu/ml of microorganisms. The liquid medium was incubated at 30° C. for 48 hours, and then, the minimum inhibitory concentration (MIC) of PAGS was measured by visually determining the growth or non-growth of the microorganisms on the basis of the medium turbidity. The liquid medium used in the test was a nutrient broth (Difco). The antibiotic substance used in the test was PAGS-1, and the test results are shown in Table 22.

TABLE 22
Measurement results for MIC of PAGS against
microorganisms

| MIC | |
|---|---|
| Staphylococcus | Aspergillus |
| aureus | niger |

| | 1:0 | 3:1 | 1:0 | 3:1 |
|---|---|---|---|---|
| hexadecane | 4 | 3 | 10 | 5 |
| octadecane | 4 | 2 | 10 | 4 |
| eicosane | 4 | 2 | 10 | 4 |
| 1-eicosanol | 4 | 2 | 10 | 2 |
| 1-pentadecanol | 4 | 2 | 10 | 3 |
| dibutyl phthalate | 4 | 2 | 10 | 4 |
| dioctyl phthalate | 4 | 2 | 10 | 3 |
| diisononyl | 4 | 2 | 10 | 2 |
| phthalate | | | | |
| dicyclohexyl | 4 | 2 | 10 | 4 |
| phthalate | | | | |
| methyl palmitate | 4 | 2 | 10 | 5 |
| Methyl stearate | 4 | 2 | 10 | 4 |
| Methyl linoleate | 4 | 2 | 10 | 2 |
| 6-formyl-3,4- | 4 | 1 | 10 | 2 |
| dehydro-2,8- | | | | |
| demethyl-2-(3′,6′- | | | | |
| dienyl-8′-hydroxy- | | | | |
| 4′-methylnonane)- | | | | |
| 2H-1-benzopyrane | | | | |

### INDUSTRIAL APPLICABILITY

As can be seen from the foregoing, the environmental friendly Sargassum-derived antifouling agent according to the present invention is harmless to environment, has antifouling activity against a broad spectrum of fouling organisms, can be extracted from nature, resulting in a relative reduction in production cost as compared with the existing antifouling substances, and can effectively prevent the pollution of marine environment caused by the use of toxic antifouling agents, such as TBT.

1-16. (canceled)

17. An antifouling agent comprising as active ingredient(s), one or more compound(s) selected from the group consisting of hexadecane, octadecane, eicosane, 1-eicosanol, 1-pentadecanol, dibutyl phthalate, dioctyl phthalate, diisononyl phthalate, dicyclohexyl phthalate, methyl palmitate, methyl stearate, methyl linoleate and 6-formyl-3,4-dehydro-2,8-dimethyl-2-(3′,6′-dienyl-8′-hydroxy-4′-methylnonane)-2H-1-benzopyrane.

18. The antifouling paint of claim 17, wherein the one or more compound(s) is(are) selected from the group consisting of hexadecane, octadecane, eicosane, 1-eicosanol, 1-pentadecanol, dibutyl phthalate, dioctyl phthalate, diisononyl phthalate, dicyclohexyl phthalate, methyl palmitate, methyl stearate, methyl linoleate and 6-formyl-3,4-dehydro-2,8-dimethyl-2-(3′,6′-dienyl-8′-hydroxy-4′-methylnonane)-2H-1-benzopyrane.

19. The antifouling paint of claim 7, wherein the one or more compound(s) is(are) selected from the group consisting of methyl palmitate, methyl stearate and methyl linoleate.

20. An antifouling agent containing as active ingredients one or two or more compounds selected from the following compounds:
one or two or more linear hydrocarbon-containing compounds having 15 carbons selected from the group consisting of hexadecane, octadecane, eicosane, 1-eicosanol and 1-pentadecanol;
one or two or more ester compounds selected from the group consisting of dibutyl phthalate, dioctyl phthalate, diisononyl phthalate, dicyclohexyl phthalate, methyl palmitate, methyl stearate and methyl linoleate; and
6-formyl-3,4-dehydro-2,8-dimethyl-2-(3′,6′-dienyl-8′-hydroxy-4′-methylnonane)-2H-1-benzopyrane compound.

21. A 6-formyl-3,4-dehydro-2,8-dimethyl-2-(3′,6′-dienyl-8′-hydroxy-4′-methylnonane)-2H-1-benzopyrane compound which has antifouling property.

22. A method of preparing a 6-formyl-3,4-dehydro-2,8-dimethyl-2-(3′,6′-dienyl-8′-hydroxy-4′-methylnonane)-2H-1-benzopyrane compound, the method comprising the steps of:
crushing Sargassum confusum as marine algae to make Sargassum confusum powder;
extracting the Sargassum confusum powder with one solvent selected from the group consisting of hexane, ethyl acetate and methanol and collecting the supernatant; and
vacuum-concentrating the collected supernatant.

23. An environmental friendly antifouling paint comprising a resin, a solvent, a pigment, an antifouling substance and other additives, in which the antifouling substance is one or a mixture of two or more compounds selected from the group consisting of hexadecane, octadecane, eicosane, 1-eicosanol, 1-pentadecanol, dibutyl phthalate, dioctyl phthalate, diisononyl phthalate, dicyclohexyl phthalate, methyl palmitate, methyl stearate, methyl linoleate, and 6-formyl-3,4-dehydro-2,8-dimethyl-2-(3′,6′-dienyl-8′-hydroxy-4′-methylnonane)-2H-1-benzopyrane.

24. The antifouling paint of claim 23, wherein the content of the resin is 2-20% by weight based on the total weight of the antifouling paint.

25. The antifouling paint of claim 23, wherein the content of the solvent is 10-30% by weight based on the total weight of the antifouling paint.

26. The antifouling paint of claim 23, wherein the content of the pigment is 20-40% by weight based on the total weight of the antifouling paint.

27. The antifouling paint of claim 23, wherein the content of the antifouling substance is 3-40% by weight based on the total weight of the antifouling paint.

28. The antifouling paint of claim 23, which additionally contain a booster for increasing antifouling performance in an amount of 1-7% by weight, the booster being one or two or more selected from the group consisting of zinc pyrithione, copper pyrithione, polyhexamethylguanidine phosphate, 2,4,5,6-tetrachloro-isophthalonitrile, 3-(3,4-dichlorophenyl)-1,1-dimethylurea, 2-methylthio-4-terbutylamino-6-cyclopropylamino-s-triazine, zinc ethylenebisdithiocarbamate, manganese ethylenebisdithiocarbamate, 2-n-octyl-4,5-dichloro-2-methyl-4-isothiazoline-3-one, 2-(thiocyanomethylthio)benzothiazole, 2,3,5,6-tetrachloro-4-(methylsulphonyl)pyridine, 3-iodo-2-propynyl butylcarbamate, diiodomethyl-p-tolylsulfone, 1,2-benzoisothiazolin-3-one, 2-methylthio-4-tert-butylamino-6-cyclopropylamino-s-triazine, 2-(4-thiocyanomethylthio)benzothiazole, 2-n-octyl-4-isothiazolin-3-one, N-(fluorodichloromethylthio)-phthalimide, N-dichlorofluoromethylthio-N′,N′-dimethyl-N-p-tolylsulfamide, N,N-dimethyl-N′-phenyl-(fluorodichloromethylthio)-sulphamide, zinc(2-pyridylthio-1-oxide), copper (2-pyridylthio-1-oxide) and silver compounds.

29. The antifouling paint of claim 23, wherein the content of the other additives is 1-5% by weight based on the total weight of the antifouling paint.

30. A biocide containing as active ingredient(s), one or more compound(s) selected from the group consisting of hexadecane, octadecane, eicosane, 1-eicosanol, 1-pentadecanol, dibutyl phthalate, dioctyl phthalate, diisononyl phthalate, dicyclohexyl phthalate, methyl palmitate, methyl stearate, methyl linoleate and 6-formyl-3,4-dehydro-2,8-dimethyl-2-(3′,6′-dienyl-8′-hydroxy-4′-methylnonane)-2H-1-benzopyrane.
